(19) United States
(12) Patent Application Publication
(10) Pub. No.: US 2013/0078224 A1
(43) Pub. Date: Mar. 28, 2013

(54) INDUCTION/MONITORING OF ARTERIOGENESIS USING SDF1 AND PDGFB OR INHIBITION OF PHD2

(71) Applicants: Massimiliano Mazzone, Leuven (BE); Peter Carmeliet, Blanden (BE)

(72) Inventors: Massimiliano Mazzone, Leuven (BE); Peter Carmeliet, Blanden (BE)

(73) Assignees: LIFE SCIENCES RESEARCH PARTNERS VZW, Leuven (BE); VIB VZW, Gent (BE)

(21) Appl. No.: 13/637,329

(22) PCT Filed: Mar. 30, 2011
(86) PCT No.: PCT/EP2011/054936

§ 371 Date: Dec. 11, 2012

Related U.S. Application Data
(60) Provisional application No. 61/341,432, filed on Mar. 30, 2010.

(51) Int. Cl. A61K 35/28

(52) U.S. Cl. CPC A61K 35/28
USPC 424/93.21; 514/8.2

(57) ABSTRACT

The disclosure relates to the field of ischemia and how to increase tissue perfusion in ischemic tissue by cellular therapy. Specifically, the beneficial effects of myeloid (bone marrow-derived) cells with a particular arteriogenic gene expression profile are shown, and it is shown that increased arteriogenesis and perfusion is specifically due to the effects of combined PDGFB and SDF-1. The arteriogenic gene profile of the myeloid cells used for therapy can, for instance, be obtained by inhibition of PHD2.

### CROSS-REFERENCE TO RELATED APPLICATIONS

This application is a national phase entry under 35 U.S.C. §371 of International Patent Application PCT/EP2011/054936, filed Mar. 30, 2011, designating the United States of America and published in English as International Patent Publication WO 2011/121036 A2 on Oct. 6, 2011, which claims the benefit under Article 8 of the Patent Cooperation Treaty and under 35 U.S.C. §119(e) to U.S. Provisional Patent Application Ser. No. 61/341,432, filed Mar. 30, 2010.

### TECHNICAL FIELD

The disclosure relates to the field of ischemia and how to increase tissue perfusion in ischemic tissue by cellular therapy. Specifically, the beneficial effects of myeloid (bone marrow-derived) cells with a particular arteriogenic gene expression profile are shown, and it is shown that increased arteriogenesis and perfusion is specifically due to the effects of combined PDGFB and SDF-1. The arteriogenic gene profile of the myeloid cells used for therapy can, for instance, be obtained by inhibition of PHD2.

### BACKGROUND

Ischemic diseases are among the leading causes of death worldwide. Examples include coronary artery disease (ischemic heart disease or myocardial ischemia), leading to myocardial infarction or heart attack and cerebral infarction (stroke). Ischemia is also found in other diseases such as peripheral vascular disease. Typically, ischemia is the result of an occlusion of a main artery that results in insufficient perfusion and subsequent hypoxia and infarction of the dependent vascular territories. Natural processes occurring in the adult organism to prevent ischemic tissue damage include angiogenesis (i.e., sprouting or de novo growth of capillaries) and arteriogenesis (growth of preexistent collateral anastomoses into functional conductance arteries) (Buschman and Schaper, J. Pathol. 190(3):338-42, 2000). Whereas, hypoxia is the driving force for angiogenesis, increased shear stress as a result of the redistribution of blood flow via preexistent collateral pathways as a result of increased pressure gradients across these anastomoses is a key event during early phases of arteriogenesis. In fact, arteriogenesis is the most efficient form of vessel growth to restore or improve tissue perfusion upon arterial occlusion (Simons et al., Circulation 102(11):E73-86, 2000).

Vascular stenosis reduces blood supply resulting in ischemia, which causes tissue dysfunction and demise. This condition is, however, associated with the formation of new blood vessels (angiogenesis) and remodeling of preexisting collateral arterioles (arteriogenesis) that reestablish blood flow to the downstream tissue. Spontaneous angiogenesis and arteriogenesis thus attenuate local tissue ischemia and improve the clinical outcome of the disease. Upon occlusion of an artery, the blood flow is redirected into preexisting arteriolar anastomoses, causing enhanced shear stress on the endothelium of the collateral circulation. As a consequence, endothelial cells (ECs) secrete VEGF, which induces the production of monocyte chemotactic protein-1 (CCL2/MCP1) from the endothelium itself and from adjacent smooth muscle cells (SMCs), leading to monocyte recruitment (Schirmer et al., Heart 95:191-7, 2009). Once in the periarteriolar region, monocyte-derived macrophages produce growth factors that enhance the motility and proliferation of SMCs, as well as proteases that digest the extracellular matrix and provide space for new SMCs (Schirmer et al., 2009; Heil and Schaper, Circ. Res. 95:449-58, 2004). Recent studies have analyzed the functional plasticity of mononuclear phagocytes in response to different environmental cues. For instance, in cancer and atherosclerosis, macrophages generally display an “alternatively activated” (M2) phenotype, which enhances debris scavenging, angiogenesis, tissue remodeling, wound healing, and the promotion of type II immunity. On the other hand, in inflamed tissues, macrophages display a “classically activated” (M1) phenotype, which facilitates eradication of invading microorganisms and the promotion of type I immune responses. However, macrophage heterogeneity during ischemia-induced arteriogenesis has not been elucidated yet.

Although initiation of arteriogenesis by macrophages takes place in a non-hypoxic environment distant from the ischemic area (Ito et al., Circ. Res. 80:829-37, 1997; Gray et al., Arterioscler. Thromb. Vasc. Biol. 27:2135-41, 2007), some of the cytokines that stimulate arteriogenesis are under the control of the prolyl hydroxylase domain protein PHD2. PHD2 belongs to a larger family of proteins that utilize oxygen to hydroxylate the hypoxia-inducible transcription factors (HIF)-1α and HIF-2α and, thereby, target the latter for proteasomal degradation and, hence, inactivation. In hypoxic conditions, PHDs are inactive, which allows HIFs to become stabilized and mount an adaptive response to hypoxia. Besides negatively regulating HIF accumulation, PHDs display a repressive role in controlling the activity of NF-κB, a key signaling molecule for inflammation. The control of NF-κB by PHDs can be both dependent and independent from their catalytic activity and, therefore, from the oxygen tension (Chan et al., Cancer Cell 15:527-38, 2009). It has been demonstrated that HIF-prolyl hydroxylases are repressors of NF-κB activity, likely via their potential to directly hydroxylate the inhibitor of NF-κB (IκB) kinase IKKβ, which is responsible for phosphorylation-dependent degradation of IκB inhibitors and, therefore, liberation and activation of NF-κB in response to inflammatory stimuli. Alternatively, PHD3 has been shown to associate with IKKβ independently of its hydroxylase function, thereby blocking further interaction between IKKβ and the chaperone Hsp90, which is required for IKKβ phosphorylation and release of NF-κB.

As spontaneous (angiogenesis and) arteriogenesis is insufficient to fully overcome ischemia due to occlusion of the native artery in ischemic disease, therapeutically enhancing arteriogenesis has received considerable attention, particularly in cardiac ischemia. Experiments in animals suggesting that the transfer of cells derived from bone marrow (BMC) could dramatically improve cardiac function after infarction through regeneration of the myocardium (Orlic et al., Nature 410:701-705, 2001) or neovascularization (Kocher et al., Nat. Med. 7:430-436, 2001) generated tremendous excitement. Different studies have highlighted the existence of a population of endothelial progenitor cells that eventually become incorporated within the newly foimed vasculature through a differentiation process resembling embryonic vasculogenesis (Rafii, J. Clin. Invest. 105:17-19, 2000; Urbich and Dimmeler, Circ. Res. 95:343-353, 2004). The existence of endothelial progenitor cells (EPCs) has engendered much excitement and has prompted a rapid transposition of the concept of bone marrow cell transfer to the clinics. There are indeed clinical studies suggesting that this approach is feasible, safe, and potentially effective in humans (Assmus et al., Circulation 106:3009-3017, 2002; Wollert et al., Lancet 364:141-148, 2004). However, the relevance of EPC incorporation has recently been questioned by a series of experimental studies (Wagers et al., Science 297:2256-2259, 2002; Ziegelhoeffer et al., Circ. Res. 94:230-238, 2004), as well as by the results of clinical trials entailing the injection of total BM sites into ischemic tissues (Rosenzweig, N. Engl. J. Med. 355:1274-1277, 2006, and citations therein, particularly references 5-9).

It is increasingly clear that BMCs (including monocytes and macrophages) are essential for functional artery formation through chemoattraction of SMCs (Heil et al., Am. J. Physiol. Heart Circ. Physiol. 283(6):H2411-9, 2002; Bergmann et al., J. Leukoc. Biol. 80(1):59-65, 2006; Zacchigna et al., J. Clin. Invest. 118(6):2062-75, 2008). Proposed strategies to stimulate arterial development to counter ischemia and ischemic damage include bone marrow, monocyte or macrophage cell therapy (e.g., WO2002/008389, WO2010/031006), bone marrow-derived stem cell or progenitor cell transplantation (WO2004/052177, WO2006/002420, WO2008/077094, WO2008/063753), administration by gene or protein therapy of angiogenic or growth factors (Seiler et al., Circulation 104(17):2012-7, 2001; Tirziu and Simons, Angiogenesis 8(3):241-51, 2005; possibly in combination with a CXCR4 antagonist: Capoccia et al., Blood 108(7):2438-45, 2006; WO2007/047882) or NO releasing agents (Sasaki et al., Proc. Natl. Acad. Sci. U.S.A. 103(39):14537-41, 2006; WO2007/005758) or a combination of cell and factor therapy, typically by transfecting the bone marrow-derived cells with angiogenic or growth factors (WO2002/008389, WO2003/101201, WO2005/007811, WO2006/102643, WO2007/089780; Herold et al., Hum. Gene Ther. 15(1):1-12, 2004; Herold et al., Langenbecks Arch. Surg. 391(2):72-82, 2006). Despite these different approaches, the lack of real clinical success using these approaches highlights the need for improved therapies.

### DISCLOSURE

The invention is based on the surprising finding that myeloid (i.e., bone marrow-derived) cells haplodeficient for PHD2 can recapitulate the effects seen for systemic PHD2 inhibition and treat ischemia by enhancing collateral perfusion (see PCT/EP2010/050645). This is due to a polarization of the macrophages toward an M2 phenotype, associated with increased arteriogenic gene expression. More specifically, it is shown herein that the increased arteriogenic profile of these myeloid cells is critically dependent on the combined increased expression and secretion of PDGFB and SDF1 by these cells. The combined, but not the separate, action of these proteins enhances smooth muscle cell (SMC) migration and resulting vessel maturation.

Accordingly, in a first aspect, a pharmaceutical composition is provided comprising both the growth factor PDGFB (platelet-derived growth factor subunit B) and the chemokine SDF-1 (stromal cell-derived factor-1). The composition may be provided with the isolated proteins, as myeloid cells with increased expression of these proteins, or a combination of both. Accordingly, an isolated myeloid cell population is provided, characterized by increased levels of arteriogenic gene expression as compared to a control myeloid cell population. The arteriogenic genes whose expression is increased include at least Tie2 (the endothelial-specific receptor tyrosine kinase 2), SDF-1 and PDGFB. Other arteriogenic genes of which the expression may be increased include one or more selected from Arg1, CXCR4, TGF-β, HGF, CCR2, MMP2, FIZZ and Neuropilin-1, more particularly selected from Arg1, HGF, TGF-β, CXCR4, CCR2, and neuropilin-1. While these genes are known to be arteriogenic (e.g., Grunewald et al., Cell 124(1):175-89, 2006; Carr et al., Cardiovasc. Res. 69(4):925-35, 2006; Tressel et al., Arterioscler. Thromb. Vasc. Biol. 28(11):1989-95, 2008; Hirschi et al., J. Cell. Biol. 141(3):805-14, 1998; Zacchigna et al., 2008 (see above); Schaper, Basic Res. Cardiol. 104:5-21, 2009), it is striking that several of them are also associated with M2 (anti-inflammatory or alternatively activated) macrophages (Futamatsu et al., Circ. Res. 96(8):823-30, 2005; Martinez et al., J. Immunol. 177(10):7303-11, 2006) and the myeloid cell population could thus also be characterized as a M2 cell population. In line with this, the myeloid cell population can alternatively or concomitantly be described as a population with decreased levels of M1 inflammatory genes as compared to a control myeloid cell population. Particularly envisaged M1 inflammatory genes that are down-regulated include one or more of IL-1β, IL-6, NOS2, MCP1, TNF-α, CXCL10 and IL-12, more particularly, of IL-1β, IL-6, NOS2, TNF-α, and CXCL10. Alternative or additional genes that are down-regulated include one or more of CXCL1, CXCL2, Ang1, PIGF, Rantes, CCL17, CCL22 and MMP9, particularly one or more genes selected from CXCL1, CXCL2, Rantes, CCL17, CCL22 and MMP9.

The isolated myeloid cell population can be macrophages. Also envisaged is a population of monocytes. The population can also be provided as a bone marrow sample. Particularly envisaged are bone marrow mononuclear cells, more particularly mononuclear phagocytes. According to particular embodiments, the bone marrow sample does not contain (endothelial) progenitor cells and/or does not contain stem cells. Also suitable as myeloid cells are peripheral blood mononuclear cells, particularly peripheral blood mononuclear phagocytes.

One way of obtaining the M2 polarized cells with increased arteriogenic gene expression is by using the alternative activation pathway, as described in the art (e.g., using stimulation with IL-4 or IL-10), or by inhibiting the classical activation (typically involving, e.g., IL-1, TNF-α, microbial products or IFN-γ) (Mantovani et al., Trends Immunol. 23(11):549-55, 2002; Mantovani et al., Trends Immunol. 25(12):677-86, 2004). Interestingly, another way of obtaining the myeloid cells with increased arteriogenic gene expression is by inhibition of PHD2, particularly partial inhibition of PHD2. This inhibition can be achieved through chemical compounds (e.g., small molecule inhibitors for PHD2), siRNA against PHD2, but also via genetic inhibition of PHD2, more particularly, haplodeficiency of PHD2 in the myeloid cells or acute deletion of PHD2 in myeloid cells.

According to a further embodiment, the composition containing SDF-1 and PDGFB (such as the myeloid cell population described herein) is provided for use as a medicament. Most particularly, the composition is provided for use in prevention or treatment of ischemia. Ischemia typically is that encountered in limb ischemia, muscle ischemia, cardiac ischemia, cerebral ischemia, ischemia in reperfusion injury, liver ischemia, renal ischemia or ischemic bowel disease.

Likewise, methods are provided of preventing or treating ischemia in a subject in need thereof, comprising the steps of:
Administering to the subject a composition containing SDF-1 and PDGFB.

By administering such composition to the subject, ischemia can be prevented or treated. The composition containing SDF-1 and PDGFB can be the myeloid cell population characterized by increased levels of arteriogenic gene expression (and at least increased levels of Tie2, PDGFB and SDF-1) as compared to a control myeloid cell population as described herein.

The administration of the myeloid cell population can be done via infusion of monocytes and/or macrophages. Alternatively, administration is by bone marrow transplantation. The monocytes, macrophages and bone marrow may be autologous (from the subject self) or allogeneic (different subject from the same species).

With prevention of ischemia, it is meant that the myeloid cell population is administered to the subject before onset of ischemia, or to avoid development of worse ischemia. This may, for instance, be applicable in settings of surgery, e.g., to prevent reperfusion injury, or to limit ischemia developed in a myocardial infarction. It may also be applicable in subjects at risk of ischemic damage, e.g., diabetic or hypercholesterolemic patients (Sacco, Neurology 45:S10-4, 1995).

According to a further aspect, a viral vector is provided comprising inhibitory RNA against PHD2. This vector can be used in treatment of ischemia. Indeed, by administering the vector to a patient and allowing the vector to express the inhibitory RNA (RNAi, siRNA) in the bone marrow, the PHD2 inhibited bone marrow will display the desirable properties as described herein (i.e., expression of PDGFB and SDF-1 will increase, leading to enhanced arteriogenesis). The viral vector typically is a lentiviral or retroviral vector. One example of a PHD2 inhibitor suitable for this aspect is a siRNA specific to PHD2, such as, for instance, the shRNA described by Chan et al. (Cancer Cell. 15(6):527-38, 2009).

Thus, methods are provided of preventing or treating ischemia in a subject in need thereof, comprising the steps of:
Administering to the subject a viral vector comprising inhibitory RNA against PHD2 wherein the viral vector homes to bone marrow-derived (i.e., myeloid) cells;
Allowing the inhibitory RNA against PHD2 to be expressed, thereby preventing or treating ischemia.

The homing of viral vectors to bone marrow-derived cells can be achieved, e.g., using pre-treatment with bone marrow ECM molecules (Moritz et al., J. Clin. Invest. 93(4):1451-7, 1994). Expression of the inhibitory RNA is particularly constrained to expression in the myeloid cells whereto the vectors are homed.

It is envisaged that PHD2 inhibition will be beneficial for all disorders characterized by ischemia: as ischemia is characterized by a restriction in blood supply, the increase in perfusion following PHD2 inhibition treats the ischemia itself and not particular features of a given ischemic disorder. Nevertheless, particularly envisaged disorders in which ischemia occurs include, but are not limited to: limb ischemia or critical limb ischemia, chronic obstructive pulmonary disease, ischemia-reperfusion injury, post-operative ischemia, diabetic ischemic disease such as diabetic retinopathy, ischemic cardiovascular disease, restenosis, acute myocardial infarction, chronic ischemic heart disease, atherosclerosis, ischemic stroke, ischemic cerebral infarction, or ischemic bowel disease.

Notably, the increase in perfusion is normally due to a change in morphogenesis or shape of blood vessels, but not due to change in number of vessels. Thus, PHD2 inhibitors may be used to increase perfusion. One example of a PHD2 inhibitor is a siRNA specific to PHD2, such as, for instance, the shRNA described by Chan et al. (2009).

As it is shown herein that increased levels of SDF-1 and PDGFB near the ischemic area (or the area at risk of ischemia) prevent development of ischemia, or reduce its severity. According to a further aspect, the levels of these two factors can be used to monitor progression or development of ischemia in a subject. Accordingly, methods are provided of monitoring progression of ischemia in a subject, comprising:
determining the presence and/or levels of SDF1 and PDGFB; and/or
determining the presence and/or levels of myeloid cells with increased expression of at least Tie2, SDF1 and PDGFB arteriogenic genes as compared to a control myeloid cell population
   in a sample of the subject. In these methods, increased levels of SDF1 and PDGFB, and/or increased levels of myeloid cells with increased expression of SDF1 and PDGFB, correlate with a decrease in ischemia (or a decreased risk of developing ischemia).

The sample of the subject will typically be taken from the ischemic area or a region near the ischemic area (this applies mutatis mutandis to areas at risk of developing ischemia). This because initiation of arteriogenesis may take place in a non-ischemic environment (i.e., separate from the actual ischemic area).

### BRIEF DESCRIPTION OF THE FIGURES

FIG. 1: PHD2 HAPLODEFICIENCY ENHANCES PERFUSION AND REDUCES ISCHEMIC DAMAGE

Panel A, PHD2^{+/-} mice present increased toe perfusion (laser Doppler analysis) at 12, 24 and 48 hours after femoral artery ligation compared to WT mice (N=7-13; P<0.05). Panel B, Partial loss of PHD2 improves functional endurance (treadmill running test) twelve hours after ligation, despite comparable performance at baseline (N=5; P<0.05). Panel C, Quantification of the MRI-based oxymetry of representative micrographs of crural muscle in PHD2^{+/-} mice versus WT controls at twelve hours after ligation (N=5; P=0.02). Panel D, Pimonidazole positive area is significantly reduced in PHD2^{+/-} compared to WT mice twelve hours after ligation (N=4; P=0.03). Panel E, Quantification of the necrotic area as evaluated by H&E staining in PHD2^{+/-} soleus (as a part of the crural muscle) versus WT soleus at 72 hours after femoral artery ligation (N=8; P=0.002). Panel F, Quantification of the crural muscle viability as evaluated by 2,3,5-tripheniltetrazolium chloride (TTC) staining shows increase in PHD2^{+/-} mice at 72 hours after ischemia (N=8; P=0.0002). Panel G, The quantification of the infarcted zone (percentage of left ventricular area) shows reduced infarct size in PHD2^{+/-} mice compared to WT mice 24 hours after coronary artery occlusion (N=4-5; P=0.03). Panels H and I, Collateral vessel area (H) and density (I) are increased in PHD2^{+/-} hearts compared to WT in both remote healthy myocardium and infarct site (N=4-5; P=0.0002). Asterisks in Panels A-I denote statistical significance versus WT. Error bars in Panels A-I show the standard error of the mean (SEM); all subsequent error bars are defined similarly.

FIG. 2: PHD2 HAPLODEFICIENCY PREVENTS ISCHEMIC DAMAGE

Panel A, Quantification of the 8-OHdG+ area in WT and PHD2+/− crural muscles before and after ischemia (twelve hours). At baseline, 8-OHdG+ area is similar in both genotypes. After occlusion, however, WT crural muscles present enhanced oxidative stress, while 8-OHdG+ area in PHD2+/− muscles remains the same (N=8; P=0.02). Panel B, Quantification of BrdU+ cells in the crural muscle. Cell proliferation assessed by BrdU immunostaining indicates reduced muscle regeneration in PHD2 haplodeficient mice 72 hours post-ischemia (N=3; P=0.02). Panels C and D, Vessel density (Panel C) and area (Panel D) at baseline and after femoral artery ligation in the soleus of WT and PHD2+/− mice (N=5-8; P<0.05). Asterisk in Panels A-D denotes statistical significance (P<0.05) compared to WT. Hash signs in Panels A, C, and D denote statistical significance (P<0.05) toward baseline.

FIG. 3: ENHANCED COLLATERALIZATION IN PHD2^{+/-} MUSCLES

Panels A and B, PHD2^{+/-} mice present increased number of secondary (Panel A) and tertiary (Panel B) collateral vessels at baseline, and after ischemia (12 and 72 hours post-ischemia) (N=6-11; P<0.05). Panels C and D, PHD2 haplodeficient mice present increased collateral vessel area (Panel G) and density (Panel H) compared to WT mice (N=8-14; P<0.01). Panels E and F, Increased number of collaterals in PHD2^{+/-} hind limbs evaluated by X-ray radiography. Panel G, Quantification of micro-CT angiograms of hind limbs at baseline showing increased number of large vessels (>240 μm in diameter) in the thigh of PHD2^{+/-} versus WT mice (N=6; P=0.04). Panels H-J, Quantification of micro-CT angiograms at baseline (Panel H) showing increased number of large vessels (>200 μm in diameter) in PHD2^{+/-} hearts (Panel J) versus WT (Panel I) hearts (N=6; P=0.04). Panels K-N, Morphometric analysis of α-smooth muscle actin (αSMA) collateral vessels in non-occluded and occluded adductor muscles of WT and PHD2^{+/-} mice; Panel K, number of αSMA⁺ collateral vessels (N=12; P<0.04); Panel L, Total αSMA⁺ collateral area (N=12; P<0.05); Panel M, Mean αSMA⁺ collateral vessel area (N=12; P<0.05). Panel N, Thickness of the tunica media (N=8; P=0.04). Asterisks in Panels A-D, G, H, and K-N denote statistical significance versus WT. Hash signs in Panels A, B, L, M and N denote statistical significance compared to the baseline.

FIG. 4: PHD2 Haplodeficiency Does Not Affect Capillary Vessels

Panels A and B, Vessel density (Panel A) and total vessel area (Panel B) in the adductor of WT and PHD2+/− mice at baseline (N=8; P=NS). Panel C, Number of small vessels (<240 μm in diameter) in the thigh of non-ligated WT and PHD2+/− mice (Micro-CT angiography) (N=6; P=NS). Panel D, Vessel density in WT and PHD2+/− hearts at baseline (N=5; P=NS). Panel E, Number of small vessels (<200 μm in diameter) in WT and PHD2+/−hearts at baseline (Micro-CT angiography) (N=5; P=NS).

FIG. 5: PHD2^{+/-} Macrophages Display a Specific Phenotype

Panels A and B, Quantification of leukocytes by CD45 immunostaining (Panel A) and macrophages by F4/80 immunostaining (Panel B) in adductor sections of WT and PHD2^{+/-} mice at baseline and after femoral artery ligation (N=8-20; P=NS). Panel C, Histogram showing increased percentage of mannose receptor C, type 1⁺ (MRC1⁺) cells out of the F4/80⁺ population in PHD2^{+/-} adductors at baseline and 72 hours post-ligation (N=8; P=0.04 in baseline and N=8; P=0.03 in ischemia); MRC1⁺F4/80⁺ cells are significantly augmented in occluded WT and PHD2^{+/-} limbs compared to the baseline (N=8; P<0.001 in WT mice; N=8; P=0.03 in PHD2^{+/-} mice). Panel D, Gene expression analysis (qRT-PCR) in WT and PHD2^{+/-} peritoneal macrophages (pMØ). PHD2 haplodeficiency up-regulates some M2-like markers, whereas some other M2-like markers and all the M1-like genes tested are down-modulated (N=8-23, P<0.05). Panel E, Gene expression analysis (qRT-PCR) in F4/80⁺ tissue macrophages sorted from adductor muscles confirms increased levels of M2 markers (PDGFB, SDF1, Tie2, MMP2, Nrp1) in PHD2^{+/-} mice at baseline (N=6; P<0.03). Seventy-two hours after femoral artery occlusion, RNA expression levels of all the genes tested, except SDF1 (N=6; P=0.01), caught-up in WT macrophages (N=6; P=NS). Grey and blue bars refer respectively to WT and PHD2^{+/-} macrophages at baseline, white and black bars to WT and PHD2^{+/-} macrophages in ischemia. Data in Panels D and E are expressed as fold change relative to the WT macrophages in either baseline. Asterisks in Panels C-E denote statistical significance. Hash signs in Panels A, B, C and E denote statistical significance compared to baseline.

FIG. 6: PHD2 Haplodeficiency Does Not Modify MCP1 Expression

Panels A-C, Histograms showing comparable expression (qRT-PCR) of MCP1 (Panel A), angiopoietin-1 (Panel B) and angiopoietin-2 (Panel C) in adductor muscles of non-ligated and ligated WT and PHD2+/− mice (N=6-18; P=NS). MCP1, angiopoietin-1 and angiopoietin-2 levels increased after ligation and were comparable in both genotypes (N=6-18; P<0.005). Hash signs in Panels A, B and C denote statistical significance (P<0.005) versus baseline.

FIG. 7: Myeloid-Specific Deletion of a PHD2 Allele Prevents Ischemica Damage

Panels A and B, Heterozygous deficiency of PHD2 in myeloid cells (PHD2^{LysCre;lox/wt}; labeled as lox/wt) increases the basal number of secondary (Panel A) and tertiary (Panel B) collateral branches (assessed by gelatin bismuth-based angiography) compared to both WT (PHD2^{LysCre;wt/wt}; labeled as wt/wt) and PHD2 homozygous deficiency (PHD2^{LysCre;lox/lox}; labeled as lox/lox) (N=18; P=0.01 and P=0.02 respectively). Panels C and D, Histological quantification on adductor sections of bismuth⁺ collateral vessel area (Panel C) and density (Panel D) at baseline (N=20; P=0.01 and P=0.003, respectively). Panel E, Quantification of necrotic area (%) represented in Panels F, G and H (N=5-12; P=0.03). Panel F, Heterozygous but not homozygous loss of PHD2 in myeloid cells improves functional endurance (treadmill running test) twelve hours after ligation, despite comparable performance at baseline (N=5; P<0.05). Panels G and H, Histograms showing collateral vessel density (Panel G) and area (Panel H) of non-occluded limbs five weeks after bone marrow transplantation. PHD2^{+/-} bone marrow in WT and PHD2^{+/-} recipient mice (HE→WT and HEΔHE, respectively) increase the number of bismuth⁺ collateral vessels at baseline; WT bone marrow transplants result in a lower number of collateral branches regardless of the genotype of the recipient mice (WT→WT and WT→HE). Panel I, Quantification of ischemic necrosis 72 hours post-ischemia. Panel J, The running capacity at twelve hours after femoral artery occlusion is increased in HE→WT mice compared to controls (WT→WT). Asterisks in Panels A-F denote statistical significance toward wt/wt and lox/lox. Asterisks in Panels G-J denote statistical significance versus WT→WT.

FIG. 8: PHD2^{+/-} Macrophage-Derived SDF1 and PDGFB Promote Arteriogenesis

Panel A, Migration of primary endothelial cells (ECs) toward control medium, WT and PHD2^{+/-} macrophages. Quantification of transmigrating ECs is represented (N=8; P=NS). Panel B, Migration of primary smooth muscle cells (SMCs) toward control medium, WT and PHD2^{+/-} macrophages. Quantification of transmigrating SMCs is represented (N=16; P<0.0001). Panel C, Combined pharmacological inhibition of SDF1 pathway by AMD3100 and PDGFB pathways by imatinib reduces SMC migration toward PHD2^{+/-} macrophages (N=8; P<0.02). Panels D and E, SMC growth (Panel D) is enhanced in presence of medium conditioned by PHD2^{+/-} macrophages (N=4; P<0.001). Conversely, EC growth (Panel E) is comparable (N=4; P=NS). Panels F-J, The stimulation of SMCs with PHD2^{+/-} macrophage-conditioned medium promotes a synthetic (proliferative) phenotype characterized by reduced RNA expression of calponin-1 (Panel F), SM22α (Panel G), smoothelin (Panel H), NmMHC (Panel I), and αSMA (Panel J) (N=4; P<0.001). Panel K, The pharmacological inhibition of SDF1 and PDGFB pathways, alone or in combination, prevents SMC growth induced by PHD2^{+/-} macrophage-conditioned medium (N=4; P<0.05). Panels L and M, Combined administration of AMD3100 and imatinib more efficiently reduces the formation of secondary (Panel L) and tertiary (Panel M) collateral vessels induced in HE→WT mice (N=8; P<0.05). Asterisks in Panels B-M denote statistical significance versus WT (or WT→WT in R and S). Hash signs in Panels A and B denote statistical significance toward control medium, in Panels K and M toward the baseline. The dollar sign in Panels C, L and M denote statistical significance (P<0.01) toward the baseline and either treatment alone.

FIG. 9: Silencing of SDF1 and PDGFB in PHD2+/− Macrophages Reduces SMC Migration in Vitro

Panel A, WT and PHD2+/− macrophages were transduced with lentiviral vectors carrying a shRNA against SDF1 or PDGFB; knock-down of SDF1 or PDGFB alone partially abrogates SMC migration toward PHD2+/− macrophages, whereas the combined silencing is more effective (N=7-12; P<0.05). A scramble shRNA was used as control. Asterisk denotes statistical significance versus WT. Hash signs denote statistical significance toward scramble. Dollar signs denote statistical significance toward the baseline and either treatment alone.

FIG. 10: Tie2-Expressing Monocytes Promote Arteriogenesis in PHD2^{+/-} Mice in a NF-κB-Dependent Manner

Panel A, Quantification of Tie2⁺ infiltrating macrophages in WT and PHD2^{+/-} adductor muscle represented at baseline and 72 hours after ligation (N=8-14; P<0.04). Panel B, The number of Tie2⁺ circulating monocytes (CD115⁺Tie2⁺ double-positive cells) is increased in PHD2^{+/-} mice at baseline and 72 hours after ligation (N=6; P<0.001). Panel C, Tie2 mRNA levels in WT and PHD2^{+/-} circulating monocytes and tissue macrophages at baseline and 72 hours after femoral artery occlusion (N=4; P<0.05). Panel D, Fiber necrosis is reduced in untreated PHD2^{30 /-} Tie2:tk-BMT mice (N=6; P=0.04) but not after GVC treatment. Panels E and F, Quantification of second (Panel E) and third (Panel F) generation collateral branch arteries in WT Tie2:tk-BMT and PHD2^{+/-} Tie2:tk-BMT mice treated with saline or ganciclovir (GCV) showing reduced collateralization in PHD2^{+/-} Tie2:tk-BMT mice after GCV administration at baseline, three and seven days after ischemia (N=6; P<0.05). Panel G, NF-κB activity (luciferase reporter assay) is enhanced in PHD2^{LysCre;lox/wt} but not in PHD2^{LysCre;lox/lox} macrophages. Silencing of PHD3 unleashes NFκB in PHD2^{LysCre;lox/lox} macrophages. Panel H, NF-κB is modulated by the hydroxylase activity of PHD2 in macrophages. The electroporation of PHD2^{+/-} macrophages with a wild-type PHD2 (PHD2^{wt}) blunts NF-κB activation, whereas a PHD2 construct containing a mutation at the catalytic site (PHD2^{H313A}) is not effective (N=4; P<0.05). Panel I, PHD2^{+/-} macrophages present enhanced NF-κB activity at baseline and upon TNF-α stimulation compared to WT macrophages (N=4; P<0.05). Panel J, Histogram showing down-modulation of PHD2 in WT bone marrow-derived monocyte/macrophage cultures after stimulation with angiopoietin-1 (Ang1) or angiopoietin-2 (Ang2) while no effect in PHD2^{+/-} monocytes/macrophages (N=4; P<0.03). Panel K, Expression of PDGFB, SDF1 and Tie2 (qPCR) WT and PHD2^{+/-} macrophages upon treatment with 50 and 250 ng/ml angiopoietin-1 (N=4; P<0.03). Panel L, Histogram showing the transcript levels of PHD2 in WT and PHD2^{+/-} F4/80⁺ macrophages, sorted from adductors at baseline and in ischemia (72 hours post-ligation), in the presence or absence of an angiopoietin inhibitor (sTie), consisting of the extracellular domain of Tie2, delivered by systemic and local injection of an AAV9 (N=4; P<0,05). Asterisks in Panels A, B, and C denote statistical significance versus WT mice; asterisks in Panels D, E and F denote statistical significance versus untreated WT Tie2:tk-BMT mice. Asterisks in Panels K and L denote statistical significance toward the WT control. Hash signs in Panels A, B and C denote statistical significance compared to baseline, in Panel G, their scramble controls, and in Panels J-L, toward the WT control (baseline).

FIG. 11: Acute Deletion of One PHD2 Allele Promotes Arteriogenic Macrophages

Panel A, PHD2Rosa26CreERT;lox/wt peritoneal macrophages treated with 2 μM 4-hydroxytamoxifen (4-OHT) for 48 hours present increased expression of PDGFB, SDF1, and Tie2 resembling the phenotype of PHD2+/− macrophages (N=4; P<0.05). Panel B, WT mice transplanted with the bone marrow of PHD2Rosa26CreERT;lox/wt mice (HERosa26CreERT WT) present increased number of Tie2+ circulating monocytes (CD115+Tie2+ double-positive cells) after tamoxifen treatment (N=6; P<0.01). Panel C, Tamoxifen-treated HERosa26CreERT WT mice present increased number of 2nd and 3rd generation collateral vessels compared to untreated mice at baseline (N=10-14; P<0.01). Panel D, 72 hours after femoral artery ligation, tamoxifen-treated HERosa26CreERT WT mice present reduced necrotic area compared to untreated animals. Asterisks in Panels A, B, C, and D denote statistical significance (P<0.05) compared to untreated HERosa26CreERT WT mice.

FIG. 12: Expression of PHDS in PHD2 Heterozygous and PHD2 Null Macrophages

Panel A, RNA levels of PHD1, PHD2 and PHD3 in macrophages from PHD2LysCre;wt/wt, PHD2LysCre;lox/wt and PHD2LysCre;lox/lox (labeled as wt/wt, lox/wt and lox/lox, respectively) mice. As expected, PHD2 levels were significantly decreased in PHD2LysCre;lox/wt and PHD2LysCre;lox/lox macrophages. PHD1 and PHD3 transcript levels were higher in PHD2 heterozygous and null macrophages (N=4; P<0.01). Panel B, Quantification of PHD2 expression (qPCR) in WT and PHD2+/− bone marrow-derived macrophages upon increased concentrations (50 and 250 ng/mL) of SDF1, PDGFB, MCP1, VEGF and PlGF. These cytokines do not modulate PHD2 mRNA levels. Black bar refers to control, grey bar to 50 ng/mL, and blue bar to 250 ng/mL of the corresponding cytokine. (N=3; P=NS). Asterisks denote statistical significance (P<0.05) compared to control macrophages (PHD2LysCre;wt/wt) in Panel A and to WT control in Panel B.

FIG. 13: PHD2 Haplodeficiency Does Not Modify NF-KB Activity in EC

Panel A, NF-κB activity in WT and PHD2+/− ECs transduced with a lentiviral vector carrying a NF-κB-responsive firefly luciferase reporter before and after stimulation with TNF-α (20 ng/mL, eight hours). NF-κB activity does not differ between the genotypes at baseline, and increases to the same level upon TNF-α stimulation (N=4, P<0.05). Grey bars correspond to WT ECs, blue bars correspond to PHD2+/− ECs. Asterisk denotes statistical significance (P<0.05) compared to vehicle-treated cells.

### DETAILED DESCRIPTION

### Definitions

The invention will be further described with respect to particular embodiments and with reference to certain figures, but the invention is not limited thereto, but only by the claims. Any reference signs in the claims shall not be construed as limiting the scope. The drawings described are only schematic and are non-limiting. In the drawings, the size of some of the elements may be exaggerated and not drawn on scale for illustrative purposes. Where the term “comprising” is used in the present description and claims, it does not exclude other elements or steps. Where an indefinite or definite article is used when referring to a singular noun, e.g., “a” or “an,” “the,” this includes a plural of that noun unless something else is specifically stated.

Furthermore, the terms “first,” “second,” “third” and the like in the description and in the claims, are used for distinguishing between similar elements and not necessarily for describing a sequential or chronological order. It is to be understood that the terms so used are interchangeable under appropriate circumstances and that the embodiments of the invention described herein are capable of operation in other sequences than described or illustrated herein.

The following terms or definitions are provided solely to aid in the understanding hereof. Unless specifically defined herein, all terms used herein have the same meaning as they would to one skilled in the art of the present invention. Practitioners are particularly directed to Sambrook et al., Molecular Cloning: A Laboratory Manual, 2nd ed., Cold Spring Harbor Press, Plainsview, N.Y. (1989); and Ausubel et al., Current Protocols in Molecular Biology (Supplement 47), John Wiley & Sons, New York (1999), for definitions and terms of the art. The definitions provided herein should not be construed to have a scope less than understood by a person of ordinary skill in the art.

As used herein, the term “perfusion” refers to the process of nutritive delivery of (arterial) blood to a capillary bed in the biological tissue. Nutritive delivery particularly relates to delivery of oxygen, nutrients and/or agents carried in the blood stream.

The term “to increase” or “increasing” as used herein, especially in relation to perfusion or perfusion-related effects in the context of PHD2 inhibition, means that levels of the variable under study are higher (i.e., increased) compared to the levels of this variable in a situation where such inhibition does not take place. Likewise, “increased” in terms of gene expression of particular cells means that the levels of gene expression are higher than those in a suitable control population of cells (e.g., PHD2-inhibited cells vs. wild-type cells as control).

“Increased” in the context of perfusion does not automatically imply that the levels of this variable are increased when compared to baseline levels, as it is particularly also envisaged that better preservation of baseline levels falls under this definition. While perfusion in this case is not higher than baseline, perfusion is increased as it is higher than the same situation where no PHD2 inhibition occurs. The same applies mutatis mutandis for the term “decrease” in the context of perfusion or perfusion-related effects in PHD2 inhibition.

The term “SDF-1” as used herein refers to the gene or protein Stromal cell-derived factor 1, a stromal cell-derived alpha chemokine member of the intercrine family. The gene is sometimes also referred to as CXCL12 (for humans, Gene ID: 6387). The term “PDGFB” as used herein refers to the platelet-derived growth factor beta gene or protein (for humans, Gene ID: 5155). “PHD2” as used herein refers to the gene or protein for HIF prolyl hydroxylase 2, sometimes also indicated as EGLN1 (for humans, Gene ID: 54583).

The term “partial inhibition of PHD2” as used throughout the application refers to inhibition that takes place but is not complete. Inhibition, and partial inhibition, can occur at different levels, e.g., at the DNA, RNA or protein level, for example, using genetic knock-out, siRNA or antibodies, but regardless the mode of inhibition, it should ultimately result in less functional PHD2 activity being present. Partial inhibition of PHD2 then typically relates to a 5% to 95% decrease in functional PHD2 activity (as compared to the non-inhibited situation), a 10% to 90% decrease, a 20% to 80% decrease, a 25% to 75% decrease, a 30% to 70% decrease in PHD2 activity. According to specific embodiments, a 40% to 60% decrease in PHD2 activity, a 45% to 55% decrease in PHD2 activity or even a 50% decrease in PHD2 activity is envisaged.

“Endothelial cells” as used herein are cells that are part of the endothelium, the thin layer of cells that line the interior surface of blood vessels. Cells can be characterized as endothelial cells by the expression of specific markers, such as CD31.

The term “ischemia” as used herein refers to a restriction in blood supply due to a blood vessel-related factor. An ischemic disorder is any disorder characterized by ischemia. According to very specific embodiments, the ischemia is not ischemia as often observed in a solid tumor.

With the term “vascular remodeling” as used in the application, the remodeling of blood vessels is meant. “Remodeling” should be understood as changing the morphogenesis or shape of the blood vessels, without affecting the number of vessels, in such a way that the vessels become more functional. “Functional” in this context implies that they are less leaky, less tortuous, allow more blood flow (perfusion), have an increased diameter, or are characterized by other parameters of PHD2^{+/-} vessels as described herein. “Vascular remodeling” as used herein thus refers to the process of forming functional vessels from non-functional vessels (e.g., resulting from non-productive angiogenesis).

The invention is based on research on the specific roles of myeloid cells in arteriogenesis and which factors are most important therein. As will be detailed in the examples, by using hind limb ischemia as a model of arteriogenesis, it was found that reduced PHD2 levels in macrophages increases the production of arteriogenic cytokines, including SDF1 and PDGFB, in a NF-κB-dependent manner. An increase of Tie2-expressing monocytes/macrophages (TEMs) in the blood and tissues accounts for the superior arteriogenesis in PHD2 haplodeficient mice. As a consequence of the production of SDF1 and PDGFB by this myeloid cell population, the remodeling of collateral anastomoses is enhanced, thus conferring protection against ischemic damage. Overall, these data indicate that a reduction of PHD2 levels in monocytes/macrophages unleashes NF-κB signals that skew their polarization toward an arteriogenic phenotype by the combined secretion of SDF-1 and PDGFB.

According to a first aspect, it is envisaged that localized administration of SDF-1 and PDGFB can be used to prevent or to treat ischemia. Accordingly, pharmaceutical compositions containing SDF-1 and PDGFB are envisaged, particularly for use in medicine, most particularly for use in preventing or treating ischemia. “Preventing” as used herein refers to avoiding or delaying the onset of ischemia in subjects at risk of developing ischemia, such as, e.g., diabetic or hypercholesterolemic subjects, or subjects that will undergo surgery. This means that the compositions described herein are administered to the subject before onset of ischemia, particularly at or near the site where ischemia is expected to occur. In this way, more mature vessels can already be formed before ischemia-causing conditions (e.g., an increased number of second and third generation collateral branches can be functionally perfused), so that ischemia is less likely to occur when ischemia-causing conditions occur (e.g., surgery causing ischemia-reperfusion injury). “Treating” refers to subjects wherein an ischemic area is present; the compositions can be administered at or near the ischemic area, where they will start recruitment of, e.g., smooth muscle cells and induce maturation of preformed collateral vessels. Thus, methods to treat or prevent ischemia are provided, comprising administering a composition containing SDF-1 and PDGFB to a subject in need thereof.

A “subject” as used herein is typically a human, but can also be a mammal, particularly domestic animals such as cats, dogs, rabbits, guinea pigs, ferrets, rats, mice, and the like, or farm animals like horses, cows, pigs, goat, sheep, llamas, and the like. A subject can also be a non-mammalian vertebrate, like a fish, reptile, amphibian or bird; in essence, any animal that uses bone marrow-derived cells for arteriogenesis fulfills the definition of “subject” herein.

The compositions described herein comprise both SDF-1 and PDGFB. According to specific embodiments, the compositions consist essentially of SDF-1 and PDGFB, i.e., these are the main active ingredients. According to yet further particular embodiments, the compositions consist of SDF-1 and PDGFB in a pharmaceutically acceptable carrier. However, it is also particularly envisaged that SDF-1 and PDGFB are administered by cell therapy, i.e., by administering particular cells that show increased expression of SDF-1 and PDGFB. Accordingly, an isolated myeloid cell population with increased expression of PDGFB and SDF-1, which are secreted, is explicitly envisaged as a composition comprising PDGFB and SDF-1. Combinations of cell therapy with protein therapy (i.e., a specific myeloid cell population additionally supplemented with PDGFB and SDF-1) are also envisaged.

Administration of pharmaceutical compositions may be by any way deemed suitable by the person of skill in the art including, but not limited to, oral, inhaled, transdermal or parenteral (including intravenous, intraperitoneal, intramuscular, intracavity, intrathecal, and subcutaneous) administration. As it is particularly envisaged to use myeloid cells with increased expression of SDF-1 and PDGFB, particularly envisaged administration methods are those normally used to administer myeloid cells to a subject, such as, but not limited to, infusion of monocytes and/or macrophages, adoptive transfer and bone marrow transplantation. The bone marrow-derived cell population with increased expression of SDF-1 and PDGFB can be derived from the subject itself (autologous transfer; in this case, the cells typically undergo a manipulation ex vivo to increase expression of SDF-1 and PDGFB) or from another subject, preferably from the same species.

The compositions will typically be used in methods to treat or prevent ischemia. Ischemia can be ischemia as encountered in any tissue including, but not limited to, limb ischemia, muscle ischemia, cardiac ischemia, cerebral ischemia, ischemia in reperfusion injury, liver ischemia, and renal ischemia. Ischemia also occurs in solid tumors and can be treated as well using the methods described herein. However, according to particular embodiments, the ischemia to be treated is not ischemia in tumors, as administering growth factors and macrophages may have undesired effects in the context of tumors.

It is demonstrated herein that the combined effects of SDF-1 and PDGFB are essential to successful arteriogenesis. Thus, the myeloid cell population should have increased expression of these genes as compared to a control myeloid cell population. More particularly, it is envisaged that other arteriogenic genes are also increased in expression as well. An “arteriogenic” gene as used herein, is a gene that has a role in the arteriogenic process (i.e., the “ripening” or maturation of pre-formed blood vessels to functional vessels that can transport nutrients and oxygen). Many of these genes have been described in the art. In other words, the myeloid cell population should be polarized to the expression of arteriogenic genes. This can be done by polarization toward the M2 phenotype. Even more efficiently, myeloid cells with a TEM profile and having increased expression of both PDGFB and SDF-1 can be used in the present invention. It is particularly envisaged herein that the myeloid cells have been polarized to the desired phenotype by inhibition or partial inhibition of PHD2.

Inhibition of PHD2 can be achieved according to methods known in the art. The myeloid cells can, e.g., be treated with a PHD inhibitor, particularly a specific PHD2 inhibitor (such as, e.g., a siRNA specific for PHD2). Particularly envisaged is genetic inhibition of PHD2, e.g., as found in PHD2 haplodeficient myeloid cells, or in PHD2 knock-out macrophages and monocytes. Alternatively, acute PHD2 deletion is envisaged.

As shown in the examples, the way in which PHD2 inhibition is achieved is not essential, as long as PHD2 levels are down-regulated. As a consequence of PHD2 down-regulation, other, particularly arteriogenic, genes will be up-regulated, leading to a polarization toward an arteriogenic phenotype and increased expression of arteriogenic genes as compared to a control population. Specific examples of arteriogenic genes that are up-regulated include, of course, SDF-1 and PDGFB. Another example of an up-regulated gene that is particularly envisaged is Tie2. Other arteriogenic genes that may be up-regulated in the myeloid cells include, but are not limited to, HGF, TGFb, CXCR4, neuropilin-1, CCR2, Arg1, FIZZ and MMP2.

Contrary to cell therapies using progenitor or stem cells, the myeloid cell population is not intended for incorporation in the tissue (vasculature), but uses paracrine effects through expression of specific factors, most particularly SDF-1 and PDGFB, to recruit smooth muscle cells (SMCs) and/or pericytes to the developing vasculature in a process of arteriogenesis. According to particular embodiments, the therapy is most effective when administered before or early after occurrence of ischemia, particularly 72 hours after onset of ischemia, 48 hours after onset of ischemia, more particularly, 36 hours after ischemia, even more particularly, 24 hours after ischemia, yet even more particularly, twelve hours onset of ischemia.

It is in fact quite surprising that anti-inflammatory (M2 polarized) macrophages can assist in arteriogenesis and overcoming ischemia, as it is known that early clinical trials inhibiting the inflammatory component in myocardial infarction with methylprednisolone drastically increased mortality due to left ventricle rupture (Hammerman et al., Circulation 68(2):446-52, 1983; Mannisi et al., J. Clin. Invest. 79(5):1431-9, 1987). It was concluded that inflammation promoted by macrophages is a good thing in cardiac ischemia, especially early on after ischemia. Accordingly, it has been proposed that M1 pro-inflammatory cytokines are beneficial in treatment of ischemia and tissue repair (Kurrelmeyer et al., Proc. Natl. Acad. Sci. U.S.A. 97(10):5456-61, 2000; Gallucci et al., FASEB J. 14(15):2525-31, 2000), and cell therapy with cells pretreated with NO enhancers such as nitric oxide synthases (typical M1 markers) has been proposed (Sasaki et al., Proc. Natl. Acad. Sci. U.S.A. 103(39):14537-41, 2006; WO2007/005758).

Analogously, it is surprising that cells expressing specific arteriogenic genes such as SDF-1 (and its receptor CXCR4) can be used to treat ischemia, as AMD3100, a CXCR4 inhibitor, has been proposed to overcome ischemia by stimulating angiogenesis (Capoccia et al., Blood 108(7):2438-45, 2006; WO2007/047882), and CXCR4⁻ stem cells have been suggested for overcoming ischemia as well (WO2006/002420). Also, trapidil, a PDGF receptor antagonist, has been reported to protect against ischemic damage and reperfusion injury (Bagdatoglu et al., Neurosurgery 51(1):212-9, 2000; Sichelschmidt et al., Cardiovasc. Res. 58(3):602-10, 2003; Avian et al., J. Pediatr. Surg. 41(10):1686-93, 2006). The apparent discrepancy between these results and the present invention may, e.g., be explained by a different timeframe of administration or recruitment, or by a different mechanism. For instance, it is shown herein that the combination of SDF-1 and PDGFB is important.

Notably, the increase in perfusion observed upon administration of SDF-1 and PDGFB, or of the specific myeloid cells with increased expression of these two factors, is normally due to a change in morphogenesis or shape of blood vessels, i.e., better maturation of collaterals or arteriogenesis, but not due to change in number of vessels (neoangiogenesis).

Although “therapeutic angiogenesis” is the term generally used in the art to indicate remodeling of blood vessels to restore normal oxygenation, it is perhaps more correct to refer to “therapeutic arteriogenesis” in the present case, as it refers to maturation or widening of existing blood vessels rather than the generation of new ones. “Therapeutic angiogenesis” as used in the art is meant to cover both true angiogenesis (capillary formation) and growth or enlargement of existing vessels (arteriogenesis), see Simons et al., 2003. As used in the present application, “therapeutic angiogenesis” only intends to cover the “therapeutic arteriogenesis” part (both terms are used as synonyms here), i.e., the remodeling of blood vessels to restore normal oxygenation by changing the morphogenesis or shape of the blood vessels, but not their number. Nevertheless, despite the fact that no new blood vessels are formed, “therapeutic arteriogenesis” can also be used to restore disorders where angiogenesis has gone awry. Therapeutic angiogenesis, or therapeutic arteriogenesis (see comment above), can be used in a plethora of diseases, as suggested by Jain, 2003 and Carmeliet, 2003.

Note that “inflammatory” and “anti-inflammatory” in the context of monocytes/macrophages are used herein to indicate M1 and M2 polarization, respectively. In the art, sometimes “inflammatory monocytes” are used as a synonym for circulating monocytes (as opposed to resident macrophages), even though they can give rise to alternatively activated (M2, anti-inflammatory) macrophages (Gordon and Taylor, Nat. Rev. Immunol. 5(12):953-64, 2005). What is important to discriminate M1 versus M2 polarization is the balance between typical M1 and M2 markers (Mantovani et al., 2002; Mantovani et al., 2004), making it possible that circulating monocytes are M2 polarized and thus anti-inflammatory (see, e.g., Pucci et al., Blood 114(4):901-14, 2009). It is important to recognize that polarization toward the M1 or M2 phenotype is indeed a balance or sliding scale: a M2 macrophage may express some M1 markers (albeit to a lesser extent) and will typically not express all M2 markers simultaneously, and vice versa. Thus, expression of M1 or M2 markers (see Mantovani et al., 2002, and Mantovani et al., 2004) is best evaluated in comparison with a control myeloid population not polarized toward either phenotype, and/or by assessing the balance of more than one marker, particularly at least one M1 marker and at least one M2 marker (e.g., high expression of CCR2 and low expression of IL-12 is indicative of M2 phenotype; the opposite would indicate M1 polarization). Also, expression of arteriogenic markers is best compared to a control myeloid cell population.

Although it is particularly envisaged to administer the compositions (as proteins, cells or combinations thereof) to subjects in need thereof, e.g., to treat ischemia, it is also envisaged that the polarized cells are not administered, but are created in the subject, spurring the myeloid cells of the subject to secrete SDF-1 and PDGFB by polarizing them. In order to achieve such polarization, viral vectors are provided comprising inhibitory RNA against PHD2. These vectors can be used to treat ischemia. Accordingly, methods are provided of preventing or treating ischemia in a subject in need thereof, comprising the steps of:
administering to the subject a viral vector comprising inhibitory RNA against PHD2 wherein the viral vector homes to myeloid cells;
allowing the inhibitory RNA against PHD2 to be expressed in the myeloid cells, thereby preventing or treating ischemia.

By down-regulating PHD2 in the myeloid cells of the subject, these cells will also obtain an arteriogenic phenotype and express specific arteriogenic genes. Thus, apart from the protein and cell therapy described herein, gene therapy is envisaged as well. Alternatively, the gene therapy can be applied ex vivo, e.g., on myeloid cells isolated from the subject, to obtain a PHD2-inhibited (and thus arteriogenic) population of myeloid cells, which can then be administered to the subject as cell therapy.

Since it was found that the combination of SDF-1 and PDGFB is essential for successful blood vessel maturation (arteriogenesis) to prevent or treat ischemia, it is envisaged that the presence and/or the levels of these two proteins can be monitored to predict the evolution of ischemia. Indeed, low or decreased levels of these proteins indicate that arteriogenesis is insufficient (and, thus, the risk for ischemia is increasing), while an increase in these two proteins indicates that arteriogenesis is ongoing (and the risk for ischemia is decreased). This applies as well to myeloid cells with higher expression of SDF-1 and PDGFB: the presence of such cells can be used to monitor progression of ischemia, wherein the increased presence of these cells correlates with a decrease in ischemia (or a decreased risk of developing ischemia).

As lower levels of PHD2 automatically result in an increase of arteriogenic gene expression in myeloid cells, it may be useful to screen for SNPs in the PHD2 gene, or promoter or enhancer regions of the gene that result in lower expression of PHD2 levels (or even abolish expression of the gene). Indeed, patients with such SNPs will likely have a lower chance of ischemic complications. Introducing such SNPs in myeloid cells may be a way of inhibiting PHD2 levels in myeloid cells.

The following examples are offered to better describe the invention.

### EXAMPLES

### Experimental Methods

Animals: 129/S6 or Balb/C WT and PHD2^{+/-} mice (eight to twelve weeks old) were obtained from our mouse facility. PHD2^{+/-} and PHD2 conditional knock-out mice were obtained as previously described (Mazzone et al., Cell 136:839-51, 2009). Tie2:GFP transgenic mice were obtained from Dr. De Palma (San Raffaele Institute, Milan, Italy) (De Palma et al., Cancer Cell 8:211-26, 2005). VE-Cadherin:CreERT and PDGFRB:Cre transgenic mice were obtained from Dr. Adams (Max-Planck-Institute, Munster, Germany) (Foo et al., Cell 124:161-73, 2006; Benedito et al., Cell 137:1124-35, 2009). IKKb conditional knock-out mice were obtained from Dr. Karin (UCSD, California) (Chen et al., Nat. Med. 9:575-81, 2003). Tie2:Cre and Rosa26:CreERT transgenic mice were purchased by the Jackson Laboratory. Housing and all experimental animal procedures were approved by the Institutional Animal Care and Research Advisory Committee of the K. U. Leuven.

Mouse Model of Hindlimb Ischemia: To induce hind limb ischemia, unilateral or bilateral ligations of the femoral artery and vein (proximal to the popliteal artery) and the cutaneous vessels branching from the caudal femoral artery side branch were performed without damaging the nervus femoralis (Luttun et al., Nat. Med. 8:831-840, 2002). By using this procedure, collateral flow to adductor muscles is preserved via arterioles branching from the femoral artery, therefore, 50% up to 60% of perfusion is preserved by this method. Two superficial preexisting collateral arterioles connecting the femoral and saphenous arteries were used for analysis. Functional perfusion measurements of the collateral region were performed using a Lisca PIM II camera (Gambro). Gelatin bismuth-based angiography was perfottned as described (Carmeliet et al., Nat. Med. 7:575-583, 2001) and analyzed by photoangiographs (Nikon D1 digital camera). Collateral side branches were categorized as follows: second-generation collateral arterioles directly branched off from the main collateral, whereas third-generation collateral arterioles were orientated perpendicularly to the second-generation branches. The number of secondary and tertiary collateral arterioles was counted. After perfusion-fixation, the muscle tissue between the two superficial collateral arterioles (adductor) was post-fixed in 2% paraformaldehyde (PFA) and paraffin-embedded, and were morphometrically analyzed. An endurance treadmill-running test was perfatmed at 12, 24, 48, and 72 hours after bilateral femoral artery ligation.

Mouse Model of Myocardial Infarction: Myocardial infarction (MI) was induced by permanent ligation of the left anterior descending coronary artery. To this end, animals were anesthetized with pentobarbital (100 mg/kg i.p.), fixed in the supine position and the trachea was intubated with a 1.1 mm steel tube. Positive pressure respiration (1.5-2 ml, 70 strokes/minute) was started and the left thorax was opened in the fourth intercostal space. All muscles overlying the intercostal space were dissected free and retracted with 5-0 silk threads; only the intercostal muscles were transected. After opening the pericardium, the main left coronary artery, which was clearly visible, was ligated just proximal to main bifurcation, using 6-0 silk and an atraumatic needle (Ethicon K801). Infarction was evident from discoloration of the ventricle. The thorax was closed and the skin sutured with 5-0 silk. Animals recovered at 30° C. Sham operated animals were subjected to similar surgery, except that no ligature was placed (Lutgens et al., Cardiovasc Res. 41:473-9, 1999). Gelatin bismuth-based angiography was perfouned 24 hours after ligation and hearts were then collected in 2% PFA.

Oxymetry: Oxygen tension (pO₂) in lower limb was measured using ¹⁹F-MRI oximetry in non-ligated and ligated legs twelve hours after femoral artery ligation. The oxygen reporter probe hexafluorobenzene (HFB) was injected directly into the crural muscle. MRI was performed with a 4.7T (200 MHz, ¹H), 40 cm inner diameter bore system (Bruker Biospec). A tunable ¹H/¹⁹F surface coil was used for radiofrequency transmission and reception (Jordan et al., Magn. Reson. Med. 61:634-8, 2009).

Histology, Immunostaining and Morphometry: Adductor crural muscles and hearts were dissected, fixed in 2% PFA, dehydrated, embedded in paraffin and sectioned at 7 μm thickness. Area of necrotic tissues in the crural muscle was analyzed by Hematoxylin & Eosin (H&E) staining. Necrotic area was defined as the percentage of area that includes necrotic myocytes, inflammatory cells, and interstitial cells, compared to the total soleus area. Infarct size was measured in desmin-stained hearts 24 hours after ischemia as previously described (Pfeffer et al., Circ. Res. 44:503-12, 1979). After deparaffinization and rehydration, sections were blocked and incubated overnight with primary antibodies: rat anti-CD31, dilution 1/500 (BD-Pharmingen), mouse anti-aSMA, dilution 1/500 (Dako), rat anti-F4/80, dilution 1/100 (Serotec), rat anti-Mac3, dilution 1/50 (BD-Pharmingen), rat anti-CD45, dilution 1/100 (BD-Pharmingen), goat anti-MRC1, dilution 1/200 (R&D Systems), rat anti-Tie-2, dilution 1/100 (Reliatech), rabbit anti-desmin dilution 1/150 (Cappel). In order to analyze capillary density and area, images of CD31 stained sections of entire soleus were taken at 40×. In order to measure bismuth-positive vessel density and area, H&E-stained paraffin sections were analyzed and vessels filled with bismuth gelatin (black spots) were taken in account. Images of the entire soleus were acquired at 20× for this analysis. The values in the graph represent the averages of the mean vessel density and area per soleus muscle. The same method was used to quantify vessel capillaries and collateral branches in cardiac tissues. Collateral arteries were defined by their luminal area (>300 μm²) (Luttun et al., 2002, as above). Density and area were measured by using a KS300 (Leica) software analysis. Hypoxic cells were analyzed two hours after injection of 60 mg/kg pimonidazole into operated mice. Mice were sacrificed and muscles were harvested. Paraffin sections were stained with Hypoxiprobe-1-Mab-1 (Hypoxiprobe kit; Chemicon International) following the manufacturer's instructions. Oxidative stress and proliferation rate were assessed on 7 μm-thick cryosections by using the goat anti-8-OHdG antibody, dilution 1/200 (Serotec) and the rat anti-BrdU antibody, dilution 1/300 (Serotec). Sections were subsequently incubated with appropriate secondary antibodies, developed with fluorescent dies or 3,3′-disminobenzidine (DAB, Sigma). Whole muscle viability was assessed on unfixed 2 mm-thick tissue slices by staining with 2,3,5-triphenyltetrazolium chloride (TCC). Viable area, stained in red, was traced and analyzed. Pictures for morphometric analysis were taken using a Retiga EXi camera (Q Imaging) connected to a Nikon E800 microscope or a Zeiss Axio Imager connected to an Axiocam MRc5 camera (Zeiss), and analysis was performed using KS300 (Leica). Angiograms were obtained by X-Ray and CT angiographies of hearts and legs at baseline.

Macrophage Preparation: To harvest peritoneal macrophages (pMØ), the peritoneal cavity was washed with 5 ml of RPMI 10% FBS. The pooled cells were then seeded in RPMI 10% FBS in six-well plates (2×10⁶ cells/well), twelve-well plates (1×10⁶ cells/well), or 24-well plates (5×10⁵ cells/well). After six hours of incubation at 37° C. in a moist atmosphere of 5% CO₂ and 95% air, non-adhering cells on each plate were removed by rinsing with phosphate-buffered saline (PBS). The attached macrophages were cultured in different mediums for 12 hours or 48 hours, depending on the experiments performed, as described below. When high amounts of cells were needed (analysis for HIF accumulation and NF-κB activity), macrophages were derived from bone marrow precursors as described before (Meerpohl et al., Eur. J. Immuno. 6:213-7, 1976). Briefly, bone marrow cells (2×10⁶ cells/ml) were cultured in a volume of 5 ml in a 10 cm Petri dish (non-tissue culture-treated, bacterial grade) for ten days in DMEM supplemented with 20% FBS and 30% L929 conditioned medium as a source of M-CSF. The cells obtained in those cultures are uniformly macrophages. A culture of monocytes/macrophages can be obtained when harvesting the cells at seven days after bone marrow collection (Martinat et al., J. Virol. 76:12823-33, 2002). Tamoxifen-inducible PHD2 haplodeficient pMØ (PHD2^{Rosa26CreERT;lox/wt}) were isolated as described above. After eight hours in culture (RPMI, 10% FBS), pMØ were washed twice with PBS and treated with or without 2 μM 4-hydroxytamoxifen (4-OHT, Sigma) in complete medium for 48 hours to allow Cre recombinase activity. Cells were then washed and kept in culture for other 48 hours before mRNA isolation and gene expression analysis.

Quantitative PCR Analysis: In order to investigate gene expression in pMØ, quantitative RT-PCR (qRT-PCR) was performed. After preparing pMØ, the cells were cultured in normoxic condition for twelve hours, and the RNA was extracted. To analyze the gene profile of adductor, gastrocnemius, and soleus muscle, tissues were collected at baseline or 24 hours/72 hours post-ischemia and RNA was extracted. Macrophages and ECs were freshly sorted from dissected adductors as described below and RNA was extracted. Quantitative RT-PCR was perfouned with commercially available or homemade primers and probes for the studied genes. The assay ID (Applied Biosystems, Foster City, Calif.) or the sequence of primers and probes (when homemade) are listed in Table 5. RNA levels of Tie-2, SDF1 and PDGFB after inhibition of NF-κB pathway were measured by qRT-PCR on pMØ exposed for twelve hours to 500 nM 6-amino-4-(4-phenoxyphenylethylamino)quinazoline. To evaluate PHD2 levels on monocyte/macrophage cultures, 2.5×10⁵ bone marrow-derived monocytes/macrophages (see above) were seeded in a 24-well plate in DMEM 10% FBS and stimulated with 50 ng/ml angiopoietin-1 (Peprotech) or 50 ng/ml angiopoietin-2 (Prospec). After 24 hours, cells were harvested for RNA extraction and cDNA preparation.

Protein Extraction and Immunoblot: Protein extraction was performed using 8 M urea buffer (10% glycerol, 1% SDS, 5 mM DTT, 10 mM Tris-HCl pH 6.8) as previously described (Mazzone et al., 2009, see above). Nuclear proteins were extracted in 1% SDS buffer upon cytoplasmic separation by using a hypotonic lysis buffer (10 mM KCl, 10 mM EDTA, 0.5% NP40, 10 mM HEPES, pH=8 plus phosphatase and protease inhibitors, from Roche). Signal was detected using ECL system (Invitrogen) according to the manufacturer's instructions. The following antibodies were used: rabbit anti-HIF-1α (Novus), rabbit anti-HIF-2α (Abcam) and mouse anti-vinculin (Sigma), rabbit anti-p65 (Cell Signaling), rabbit anti-p105/50 (Abcam).

Transduction and Transfection of Bone Marrow-Derived Macrophages and Lung Endothelial Cells: To express an inducible NF-κB responsive firefly luciferase reporter, commercially available lentiviral vector particles (LV) were used (Cignal Lenti NF-κB Reporter; SABiosciences). 2.5×10⁵ bone marrow-derived macrophages and 10⁵ primary lung endothelial cells (isolated as previously described (Mazzone et al., 2009, see above)) were seeded in a 24-well plate in DMEM 10% FBS or M199 20% FBS for eight hours. Cells were transduced with 10⁸ TU/ml. Eight hours after transduction, the medium wash was replaced. After 48 hours, cells were stimulated with TNFα (20 ng/mL) for eight hours and the same amount of protein extract was read in a luminometer. For PHD3 silencing, siRNA oligonucleotides were designed using the Invitrogen online siRNA design tool (world wide web at maidesigner.invitrogen.com). The following sequences (sense strands) and target positions were used: PHD3 siRNA: 5′-GCCGGCUGGGCAAAUACUAUGUCA-3′ (SEQ ID NO:1); scramble siRNA:5′-CACCGCTTAACCCGTATTGCCTAT-3′ (SEQ ID NO:2). In brief, one day after the transduction of macrophages with LV, cells were transfected using Lipofectamine 2000 (Invitrogen) according to the manufacturer's instructions. Preparation of the oligonucleotide-Lipofectamine 2000 complexes was done as followed: 25 pmol siRNA oligonucleotide (stock: 20 μM) was diluted in 50 μl Opti-MEM I reduced serum medium. Lipofectamine 2000 (1.5 μl) was diluted in 50 μl Opti-MEM I reduced serum medium and incubated for five minutes at room temperature. siRNA oligonucleotides were gently mixed with Lipofectamine 2000 and allowed to incubate at room temperature for twenty minutes to form complexes. Just before transfection, the cell culture medium was removed and cells were rinsed twice with serum-free Opti-MEM I medium. The Lipofectamine 2000-siRNA oligonucleotide complexes were added to each well in 400 μl of serum-free Opti-MEM medium for five hours. Afterwards, cells were incubated in complete medium for 48 hours at 37° C. in a CO₂ incubator and assayed for gene knockdown (qRT-PCR) and luciferase activity. To assess if the increased NFkB activity observed in PHD2^{+/-} macrophages was dependent on the hydroxylase activity of PHD2, 48 hours before transduction, 4×10⁶ bone marrow-derived macrophages were resuspended in 240 μl of Opti-MEM and were electroporated (250 V, 950 uF, ∝ Ω) with 7 μg of plasmids expressing a wild-type PHD2 (PHD2^{wt}) or a PHD2 containing a mutation at the catalytic site (PHD2^{H313A}).

Cell Migration and Viability Assays: Migration and proliferation of SMCs and ECs were assessed by using 8 μm-pore Transwell peimeable plate for migration assays and 0.4 μm-pore Transwell permeable plate for proliferation assays (Corning Life Science). To determine cell migration toward the factors secreted by pMØ, pMØ were cultured in the lower chamber for twelve hours in RPMI 1% FBS or in M-199 1% FBS (migration assay), or 48 hours in DMEM-F12 1% FBS or in M-199 1% FBS (proliferation assay). For migration assays, hCASMC (Human coronary artery smooth muscle cells; from Lonza) and HUVEC (Human umbilical vein endothelial cells; from Lonza) were starved for twelve hours in their own medium at 1% FBS and, therefore, seeded in the upper chamber (5×10³ cells in 200 μl of medium 1% FBS), with or without AMD3100 (1 μg/ml, Sigma-Aldrich, Dorset, U.K) and imatinib (2.5 μg/ml, Novartis). SMCs and HUVECs were incubated for two days or 24 hours, respectively, and migrated cells were fixed with 4% PFA, stained with 0.25% crystal violet/50% methanol and counted under the microscope. VEGF (100 ng/mL, R&D), PDGFB (100 ng/mL, R&D) or SDF 1 (100 ng/mL, R&D) were used as positive controls. For cell growth assays, RAOSMC (Rat Aortic Smooth Muscle Cells) and HUVEC were seeded on the upper chambers (5,000 cells/transwell) and cultivated with pMØ for 24 hours in DMEM-F12 1% FBS or M-199 1% FBS for RAOSMC and HUVEC cells, respectively. The cell proliferative ability was then analyzed using WST-1 Cell Proliferation Assay (Roche Applied Biosciences) according to the manufacturer instructions after 24 hours of coculture with the pMØ. Alternatively, WT and PHD2^{+/-} pMØ were seeded in the lower chamber of a Transwell and transduced with lentiviral vectors (10⁸ TU/ml; Sigma) carrying a shRNA against SDF1 or PDGFB, or a scramble control. Sixty hours after macrophage transduction, SMC migration or growth assays were performed by seeding the SMCs in the upper side of the Transwell as described above.

SMC Differentiation Assay: pMØ were seeded in a 24-well plate with DMEM F-12 5% FBS. Conditioned medium was harvested after two days and supplemented with 25 mM HEPES. RAOSMC were seeded in 24-well plates (80,000 cells/well) and incubated for five hours at 37° C. in a moist atmosphere of 5% CO₂ and 95% air. After two hours of starvation in DMEM-F12 1% FBS, SMC were stimulated with conditioned medium from WT and PHD2^{+/-} pMØ. After 24 hours, differentiation status of the SMCs was assessed by qRT-PCR.

FACS Analysis and Macrophage and Endothelial Cell Sorting: FACS analysis of circulating TEMs was performed on 200 μl of peripheral blood, harvested by eye bleeding at baseline or at three days after femoral artery ligation. Blood samples were incubated for twenty minutes at 4° C. with a rat APC-conjugated anti-CD115, a mouse PE-conjugated anti-Tie2 (eBiosciences), a rat FITC-conjugated anti-Gr1 (BD-Pharmingen). For cell sorting of adductor macrophages and ECs, the adductors were dissected, dissociated mechanically and after digested using collagenase I for 45 minutes at 37° C. For macrophage sorting, the digested cell suspension was incubated for fifteen minutes with Mouse BD Fc Block™ purified anti-mouse CD16/CD32 mAb (BD-Pharmingen) and stained with rat FITC-conjugated anti-F4/80 antibody (Serotec) for twenty minutes at 4° C. CD31⁺CD45⁻ endothelial cells were sorted from the digested adductor cell suspension after incubation with rat APC-conjugated anti-CD31 and rat FITC-conjugated anti-CD45 antibodies (BD-Pharmingen) for twenty minutes at 4° C.

Bone Marrow (BM) Transplantation and Hematological Analysis: Balb/c WT and PHD2^{+/-} recipient mice were irradiated with 7.5 Gy. Subsequently, 5×10⁶ bone marrow cells from green fluorescent protein⁺ (GFP⁺) WT or GFP⁺ PHD2^{+/-} mice were injected intravenously via the tail vein. After one week, saline or AMD3100 (5 mg/kg) was administered intravenously and saline or imatinib (50 mg/ml) was administered by oral gavage for four weeks. Femoral artery ligation, treadmill running test and bismuth angiography were performed at six weeks after bone marrow reconstitution. Red and white blood cell count was determined using a hemocytometer on peripheral blood collected in heparin with capillary pipettes by retro-orbital bleeding. To assess the effect of acute deletion of macrophage-borne PHD2 on arteriogenesis, PHD2^{Rosa26CreERT;lox/wt} bone marrows were transplanted into lethally irradiated WT recipient mice. After five weeks, transplanted mice were injected i.p. with tamoxifen (1 mg/mouse; Sigma) or vehicle for five days. TEM quantification and femoral artery ligation were performed ten days after tamoxifen treatment as explained above. The bone marrow of PHD2^{WT};Tie2:GFP or PHD2^{HE};Tie2:GFP mice was transplanted in lethally irradiated WT mice. After five weeks, mice were injected systemically (5×10¹¹ vp) and locally (5×10⁹ vp in two points of the adductor) with an AAV9 encoding the mouse extracellular domain of Tie2 fused to a flag tag (AAV9:sTie2). AAV9 encoding the human serum albumin was used as control. One week after injection of the viral vector, mice were subjected to femoral artery ligation. Blood and adductor samples were harvested at baseline and 72 hours post-ischemia and used to sort CD115⁺GFP⁺ circulating monocytes and F4/80⁺GFP⁺ macrophages.

Bone Marrow-Deriv ed, Lineage-Negativ e Hematopoietic Cell Isolation, Transduction and Transplantation: Six- to twelve-weekold WT or PHD2^{+/-} Balb/C mice were killed and their BM was harvested by flushing the femurs and the tibias. Lineage-negative cells (lin⁻ cells) enriched in hematopoietic stem/progenitor cells (HS/PCs) were isolated from BM cells using a cell purification kit (StemCell Technologies) and transduced by concentrated lentiviral vectors. Briefly, 10⁶ cells/ml were pre-stimulated for four to six hours in serum-free StemSpan medium (StemCell Technologies) containing a cocktail of IL-3 (20 ng/ml), SCF (100 ng/ml), TPO (100 ng/ml) and FLT-3L (100 ng/ml) (all from Peprotech), and transduced with two lentiviral vectors (LVs), Tie2:tk (to deplete TEMs in transplanted mice) and PGK:GFP (to assess the efficiency of BM reconstitution in transplanted mice), with a dose equivalent to 10⁸ LV-Transducing Units/ml, for twelve hours in medium containing the cytokines. After transduction, 10⁶ cells were infused into the tail vein of lethally irradiated, six-week-old, female Balb/C mice (radiation dose: 7.5 Gy).

Vector Copy Number Analysis: Transduced lin⁻ cells were cultured and collected after nine days while blood from the transplanted mice was collected at four weeks after HS/PCs tail vein injection to measure the number of integrated LV copies/cell genome (vector copy number, VCN) by qRT-PCR, as previously described (De Palma et al., Cancer Cell 14:299-311, 2008). Briefly, for vector copy number (VCN) analysis, we performed qRT-PCR using custom TaqMan assays specific for β-actin, HSV-tk or HIV-gag sequences (Applied Biosystems). Standard curves for HSV-tk (contained by Tie2:tk LV) or HIV-gag (contained by both Tie2:tk and PGK:GFP LVs) were obtained from genomic DNA samples containing known amounts of integrated LV. The VCN of genomic DNA standard curves was determined using custom TaqMan assays specific for LVs (Applied Biosystems). The SDS 2.2.1 software was used to extract raw data (CT) and to perform VCN analysis. To calculate VCN, we used the following formula: VCN=VCN_{(standard curve)} * ng of “LV of interest”/ng of β-actin, where “LV of interest” is either HIV-gag or HSV-tk. The VCN of PGK:GFP LV was obtained by subtracting the VCN of HSV-tk from the total HIV-gag VCN.

Statistics: The data were represented as mean±SEM of the indicated number of measurements. Statistical significance was calculated by t test where indicated (Prism v4.0b), with p<0.05 considered statistically significant.

### Example 1

### Generation of PHD2+/− Mice and Expression of PHD2

This was done as described before (PCT/EP2010/050645; Mazzone et al., 2009). In brief, to study its biological role in vivo, we inactivated the PHD2 gene in the germline. PHD2-deficient (PHD2^{-/-}) mice died at mid-gestation, while PHD2^{+/-} mice developed normally, were healthy, and did not exhibit vascular defects; physiological angiogenesis was also normal. PHD2 mRNA and protein were undetectable in PHD2^{-/-} embryos and present at 50% of the normal levels in healthy organs in PHD2^{+/-} mice, with minimal up-regulation of PHD3. Also, cultured PHD2^{+/-} cells expressed 50% of the normal PHD2 levels at various oxygen tensions. Consistent with previous findings that PHDs are HIF-targets and up-regulated in chronic hypoxia (Appelhoff et al., J. Biol. Chem. 279:38458-38465, 2004; Epstein et al., Cell 107:43-54, 2001; Marxsen et al., The Biochemical Journal 381:761-767, 2004, Aragones et al., Nat. Genet. 40:170-180, 2008), PHD3, and to a lesser extent PHD1, protein levels were up-regulated in PHD2^{+/-} cells, especially in normoxic conditions. As expected, PHDs were also up-regulated in WT cells in hypoxia conditions. PHD2 becomes gradually less active in hypoxia, but still retains activity at low oxygen tensions (Epstein et al., 2001). HIF-1α levels were indeed higher in PHD2^{+/-} cells at every, even low, oxygen tension; HIF-2α levels were also up-regulated, particularly in endothelial cells (ECs). By resetting their oxygen sensing curve, PHD2^{+/-} cells act as if they continuously sense lower oxygen tensions, as if they are (pre)-adapted to hypoxia.

### Example 2

### Targeting PHD2 in Ischemic Diseases

Apart from its usefulness in disorders characterized by excessive angiogenesis, such as cancer and AMD, experiments have demonstrated that PHD2 inhibition may be useful in the treatment of ischemia, i.e., in conditions where a restriction in blood supply exists (PCT/EP2010/050645). Although at first sight this may appear contradictory, the examples shown therein demonstrate that heterozygous deficiency of PHD2 results in mature and more stable pathological vessels, which is beneficial in ischemic conditions. For instance, this was evaluated in a limb ischemia model after femoral artery ligation in WT and PHD2^{+/-} mice. To induce limb ischemia, the right femoral artery was occluded distal to the branch site of the deep femoral and the popliteal artery. After 1 or 3 or 14 days, mice were perfused with fixative and bismuth-gelatin contrast medium for angiography. Collaterals in the adductor muscle were used for morphometry.

### PHD2 Haplodeficiency Preserves Tissue Perfusion and Viability in Ischemia

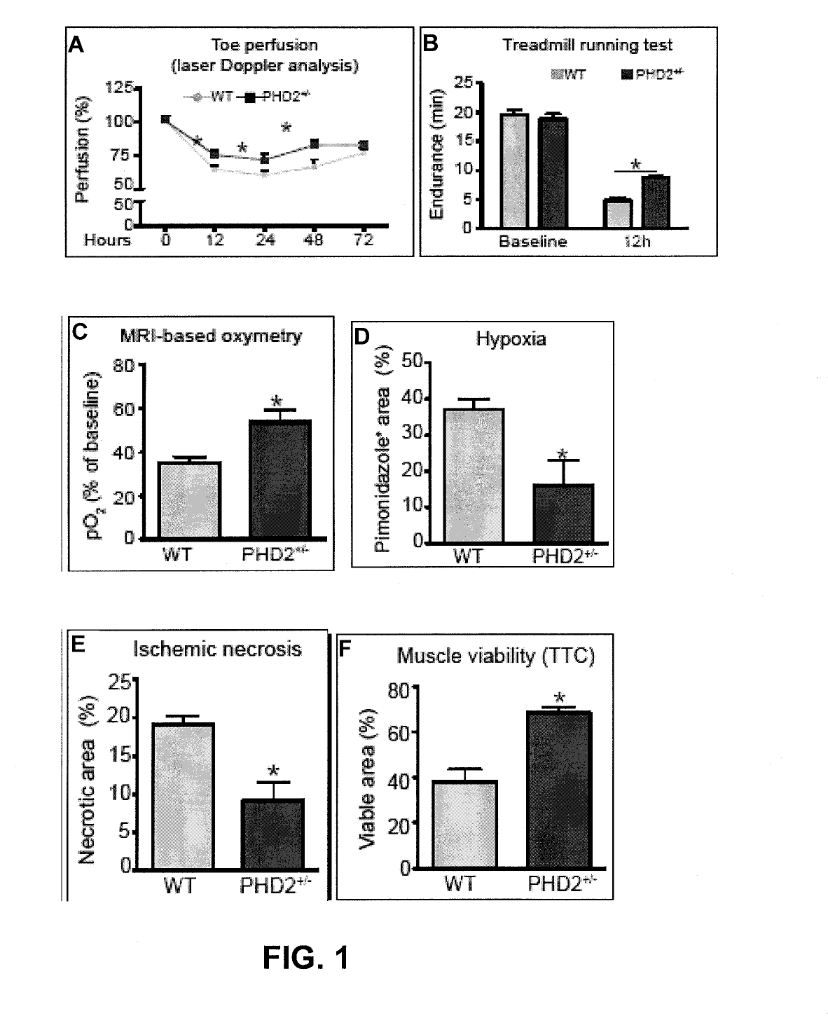

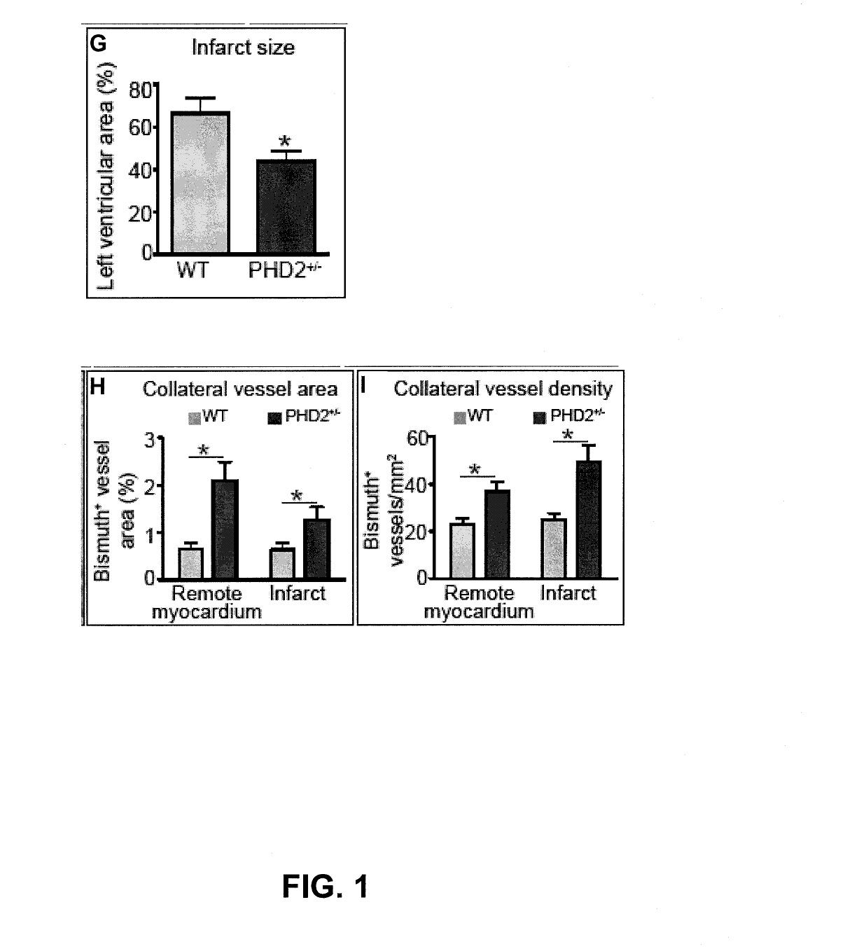

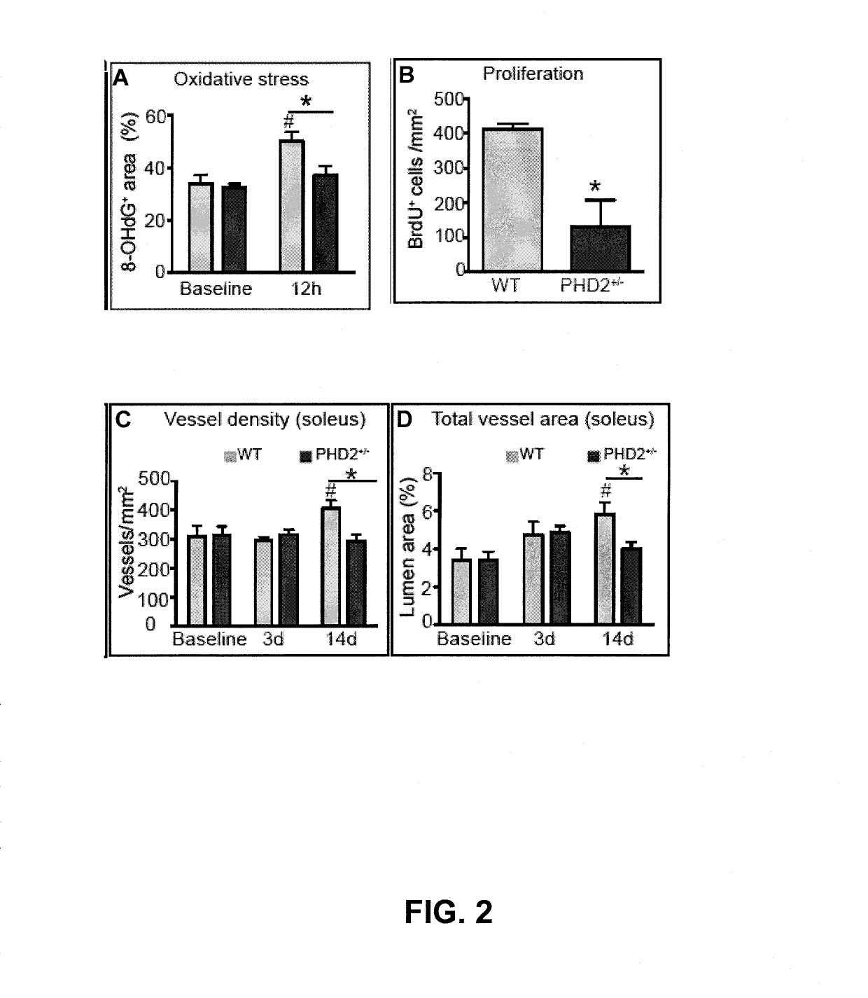

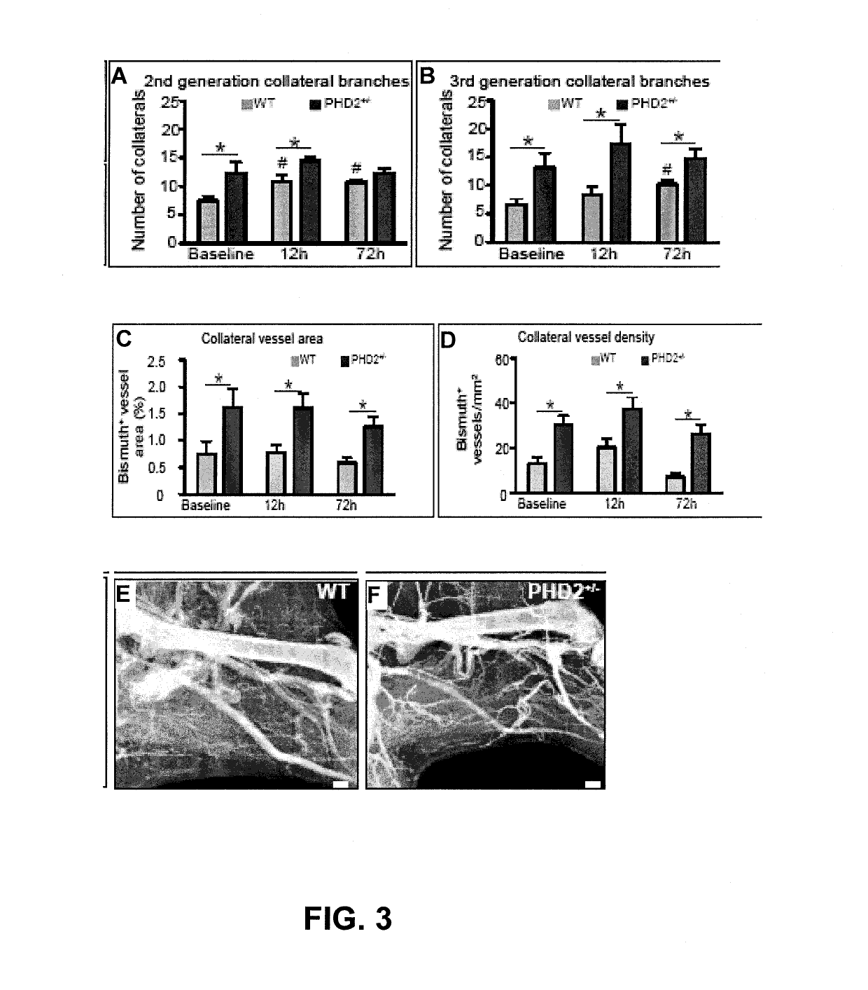

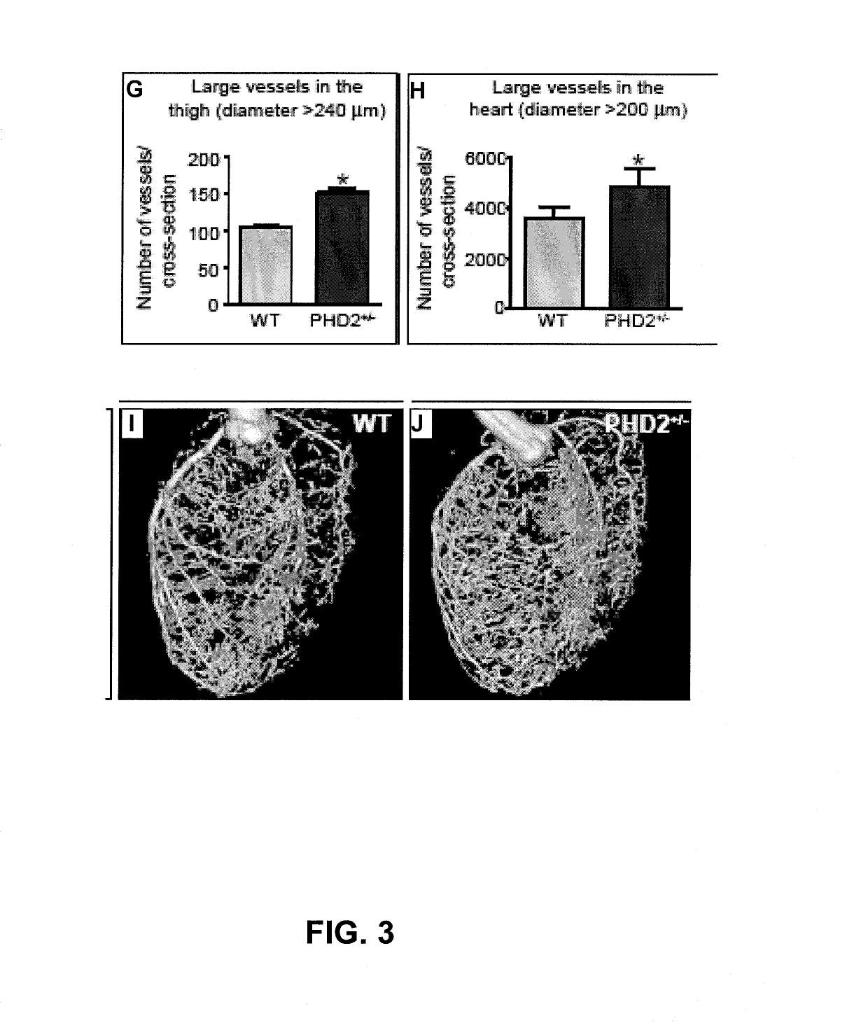

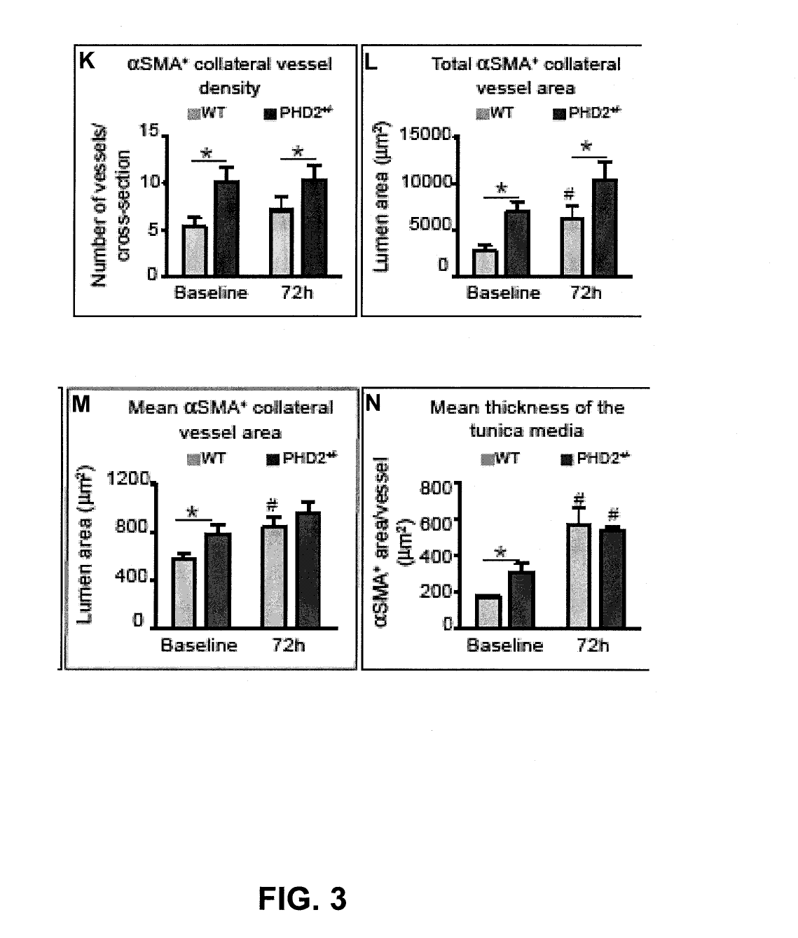

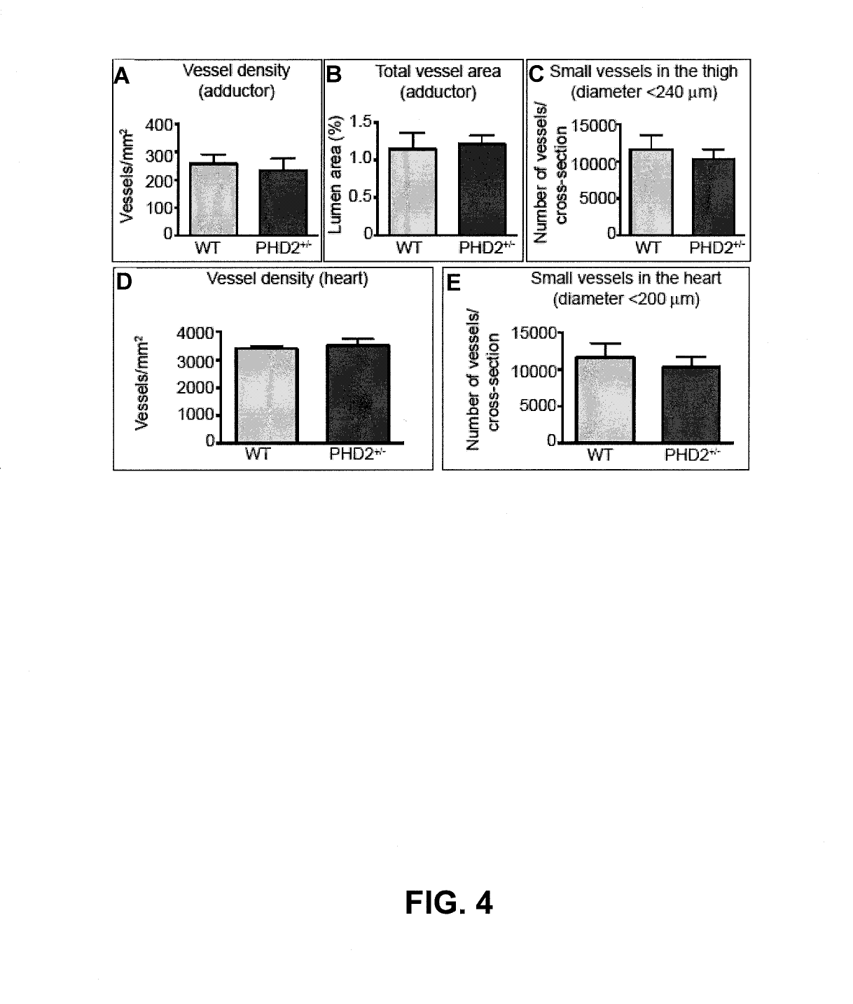

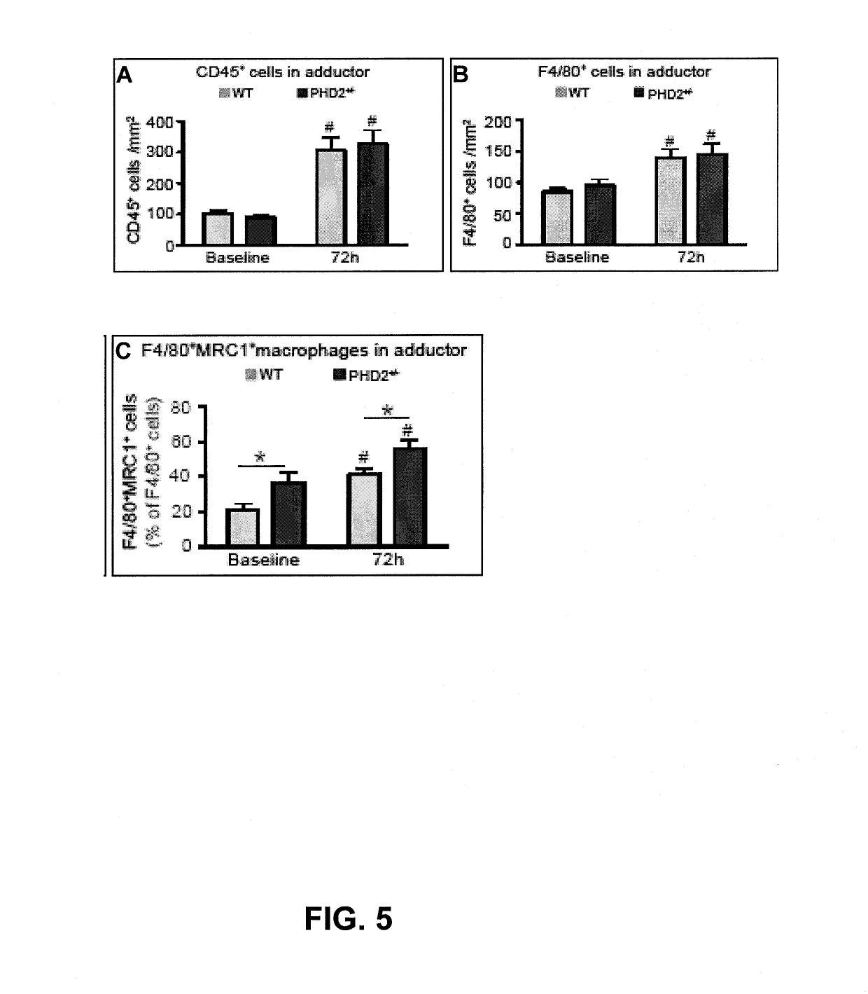

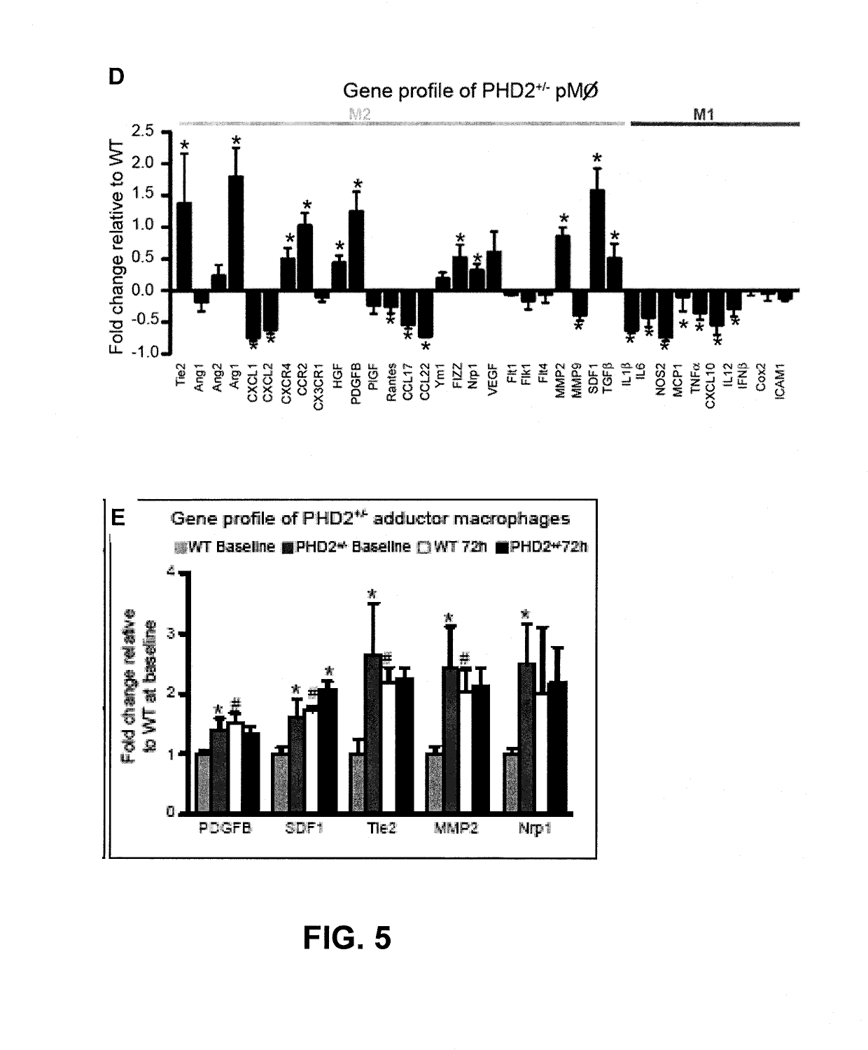

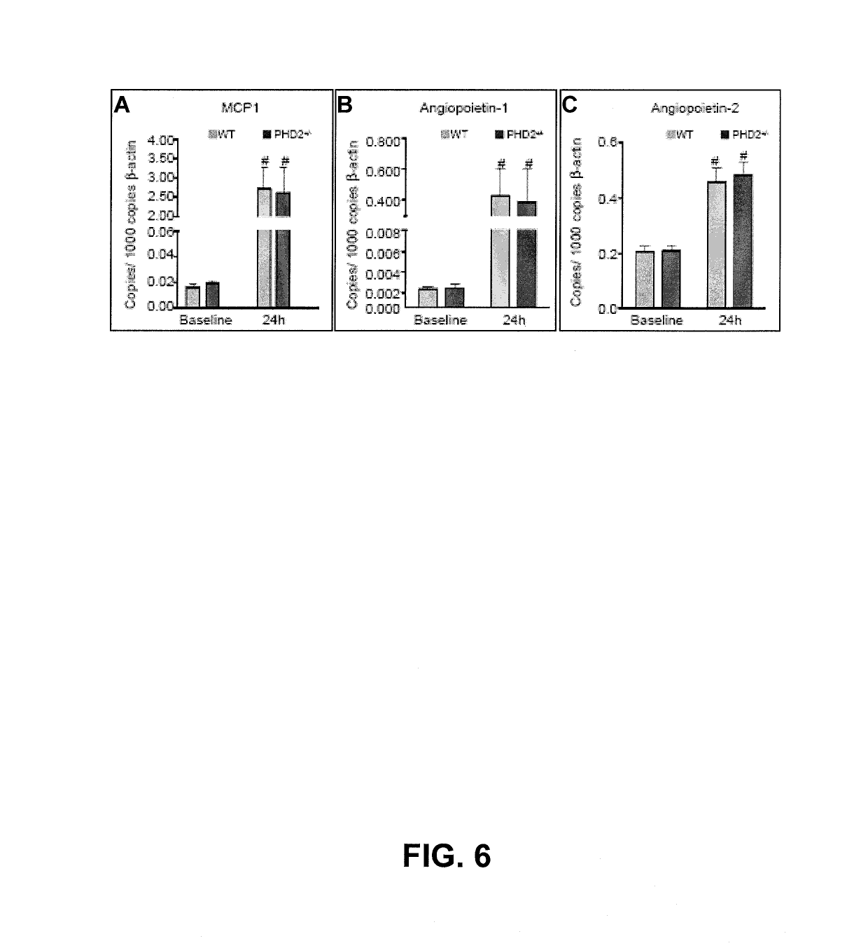

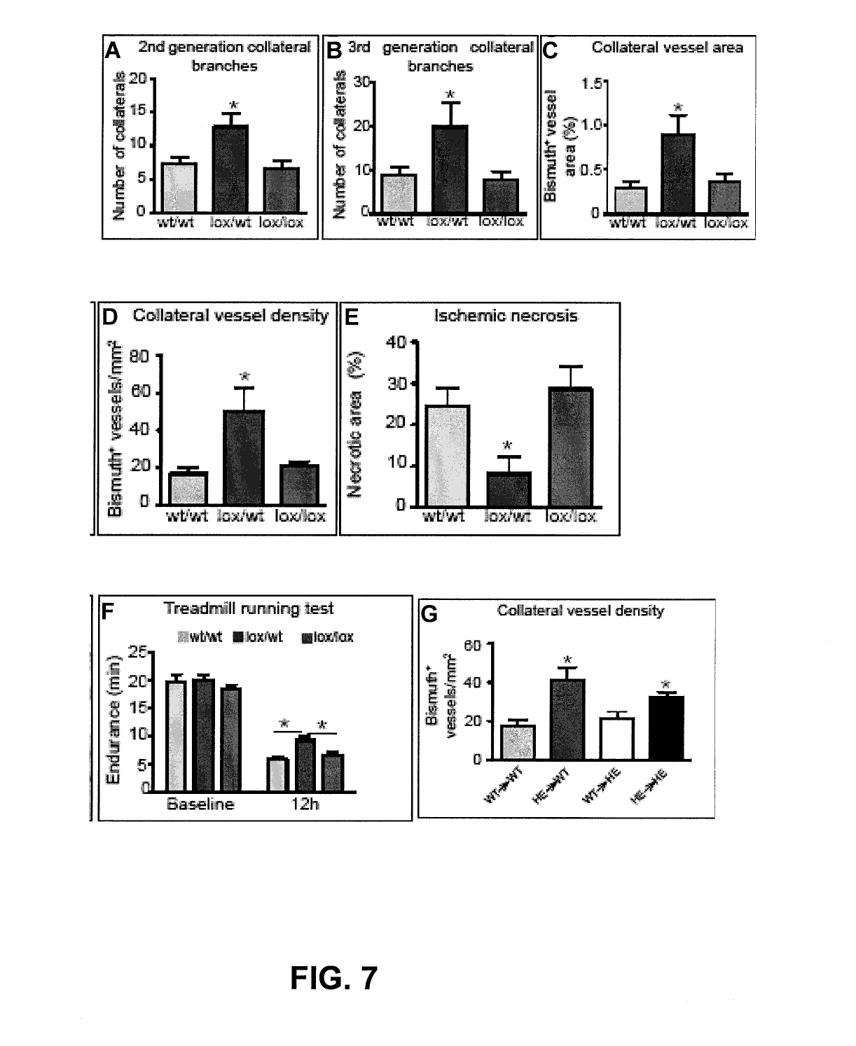

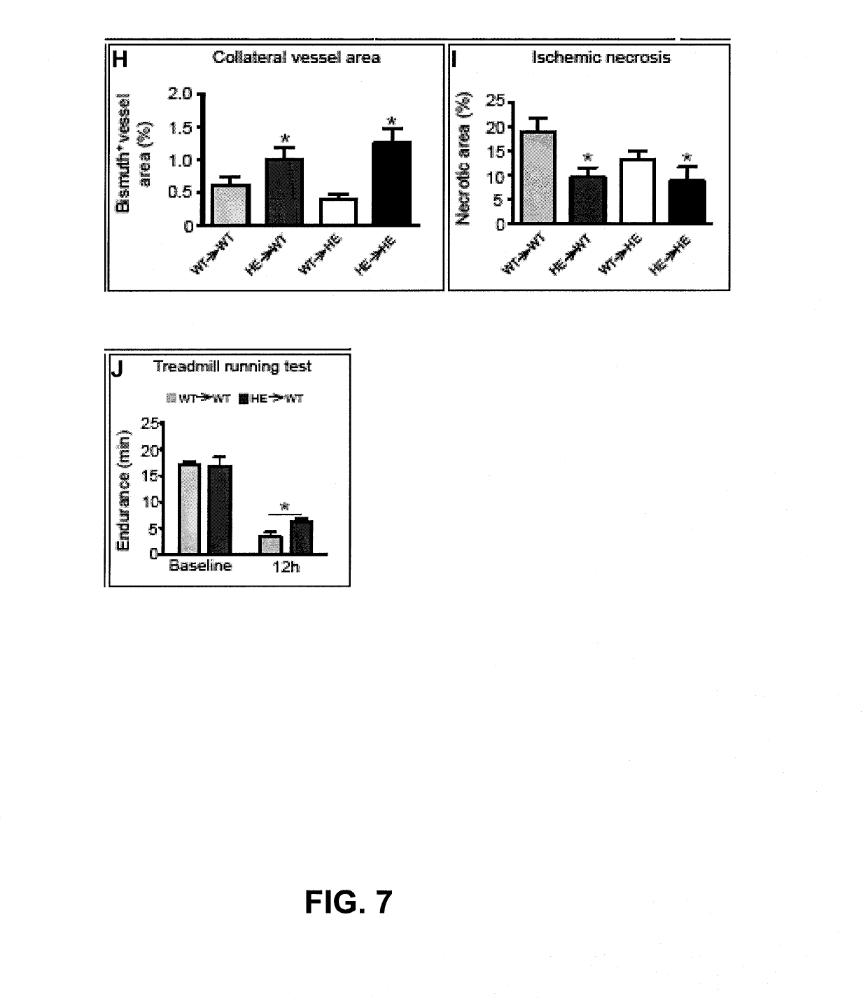

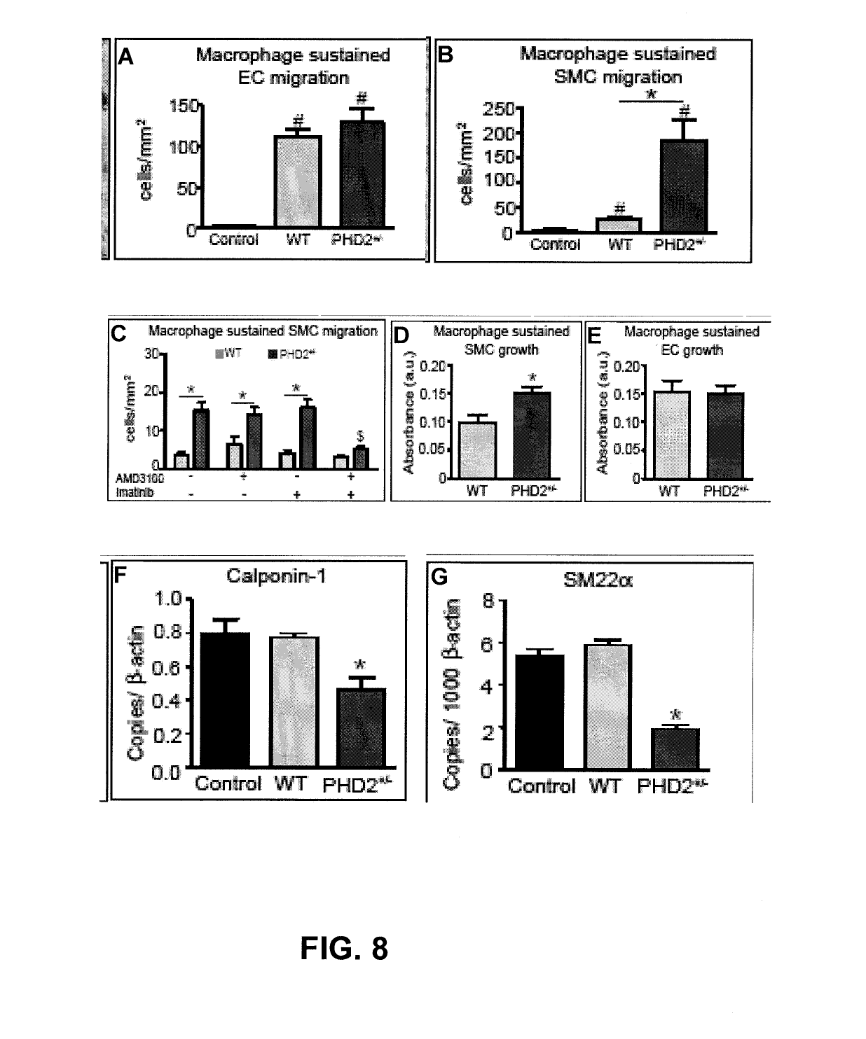

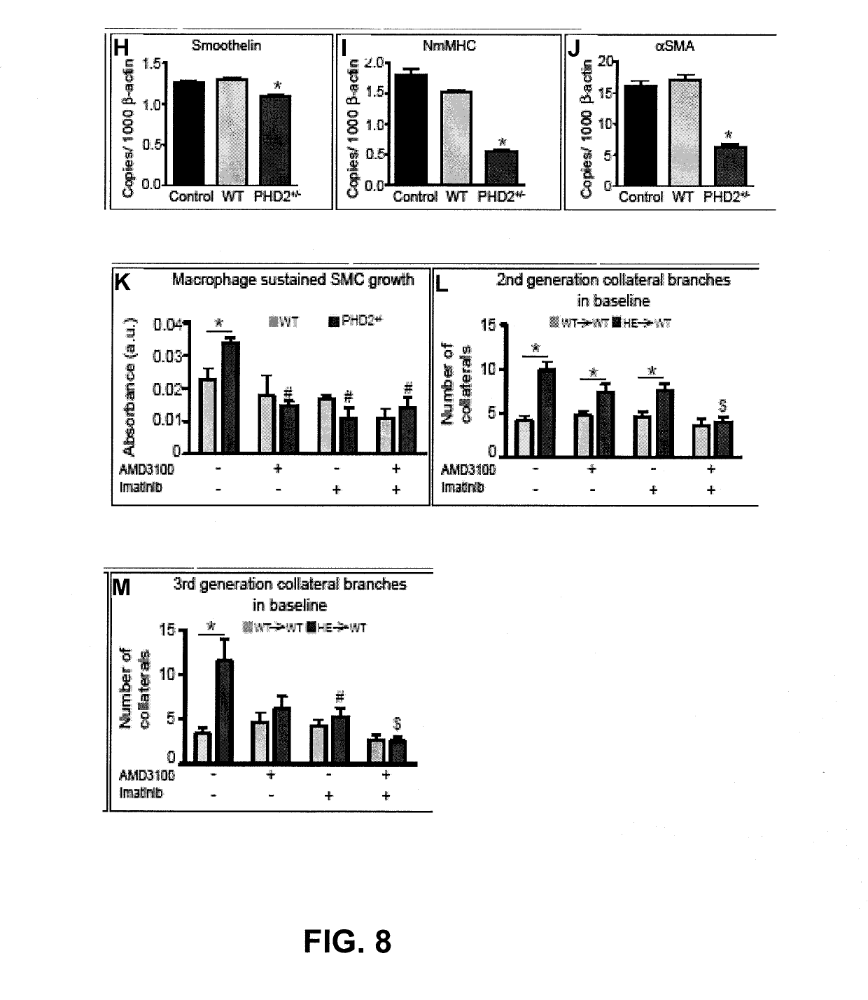

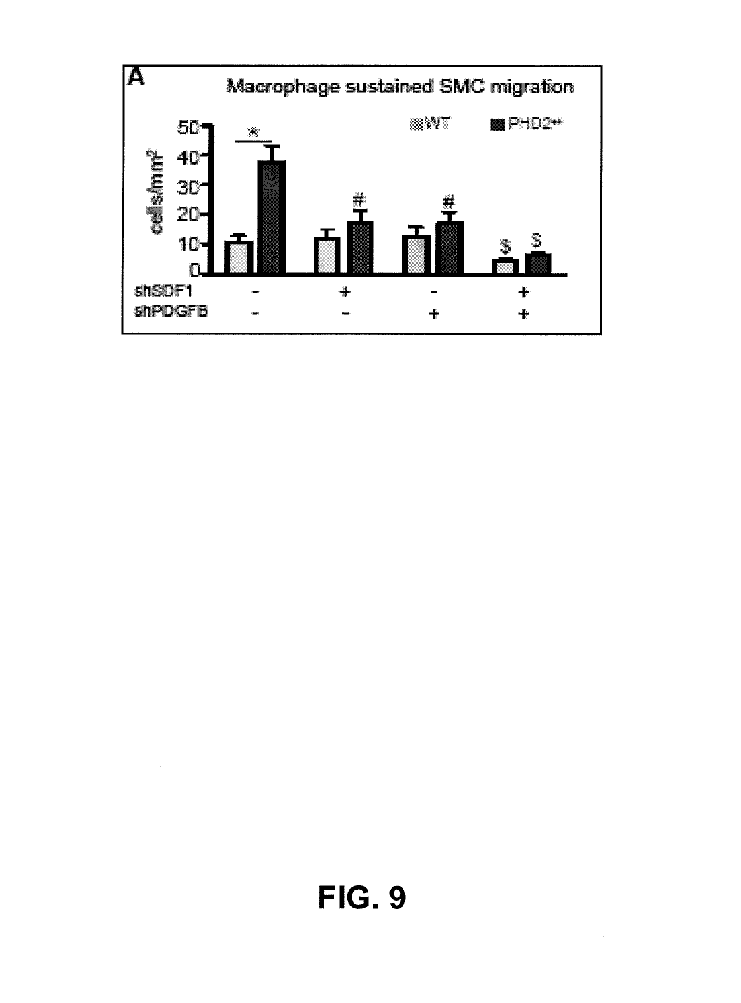

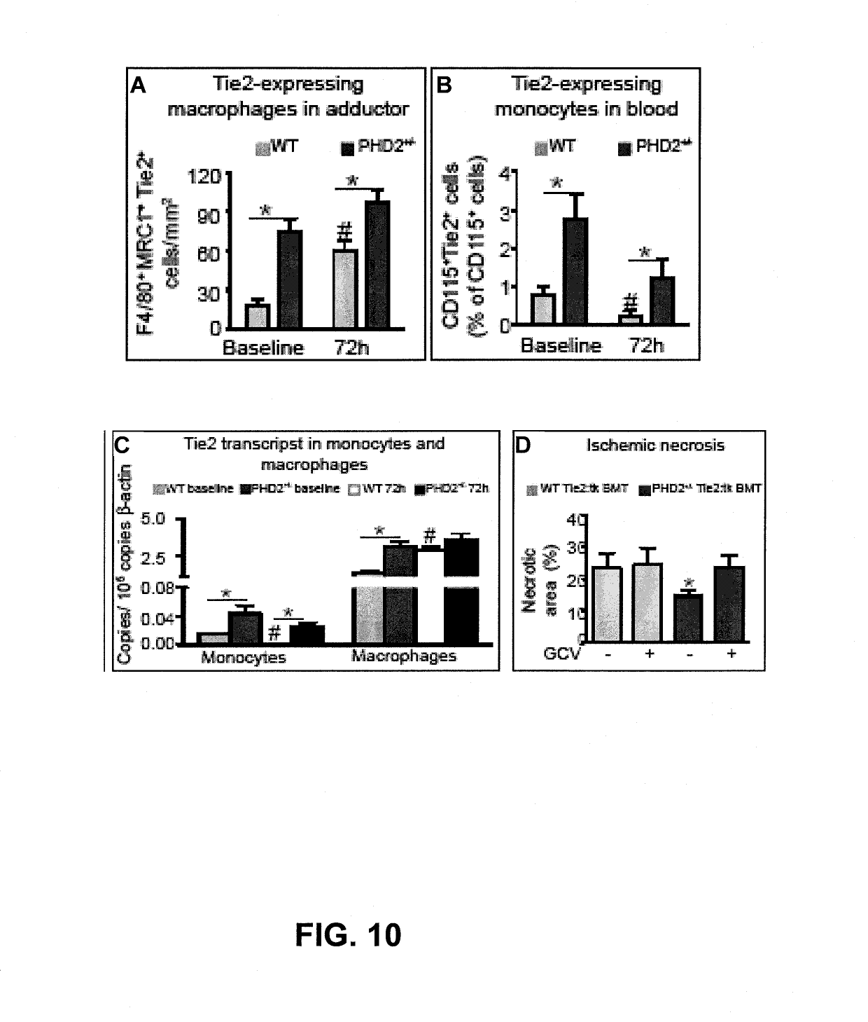

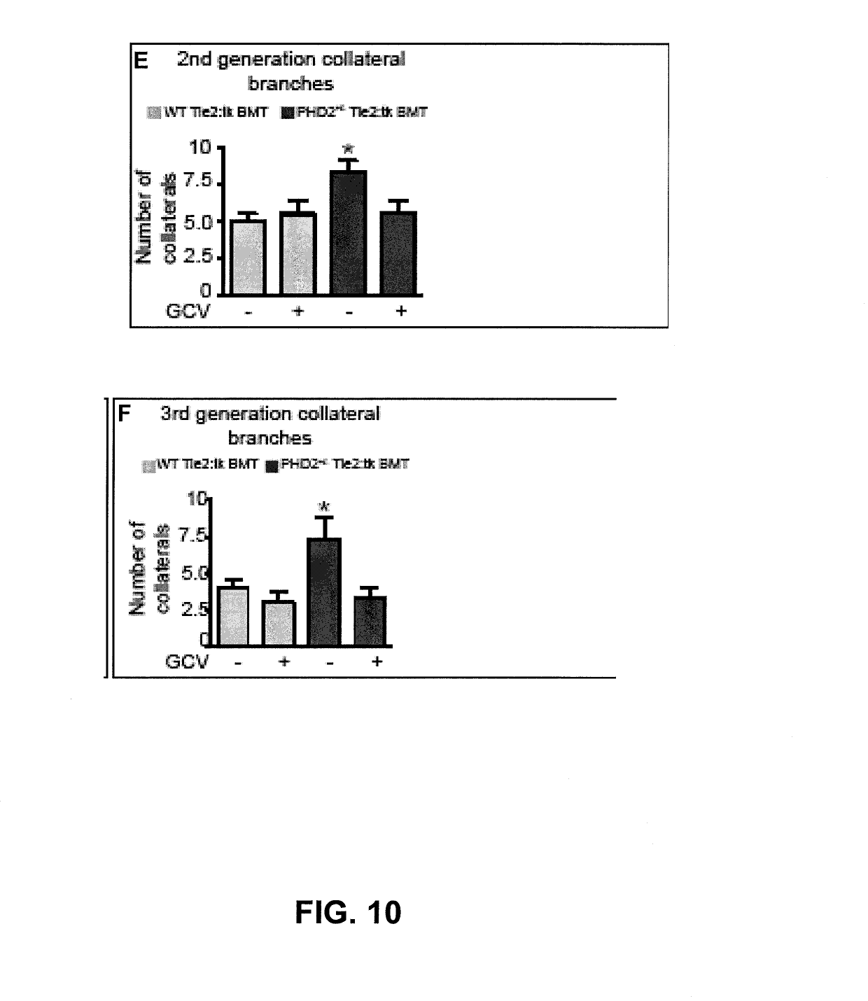

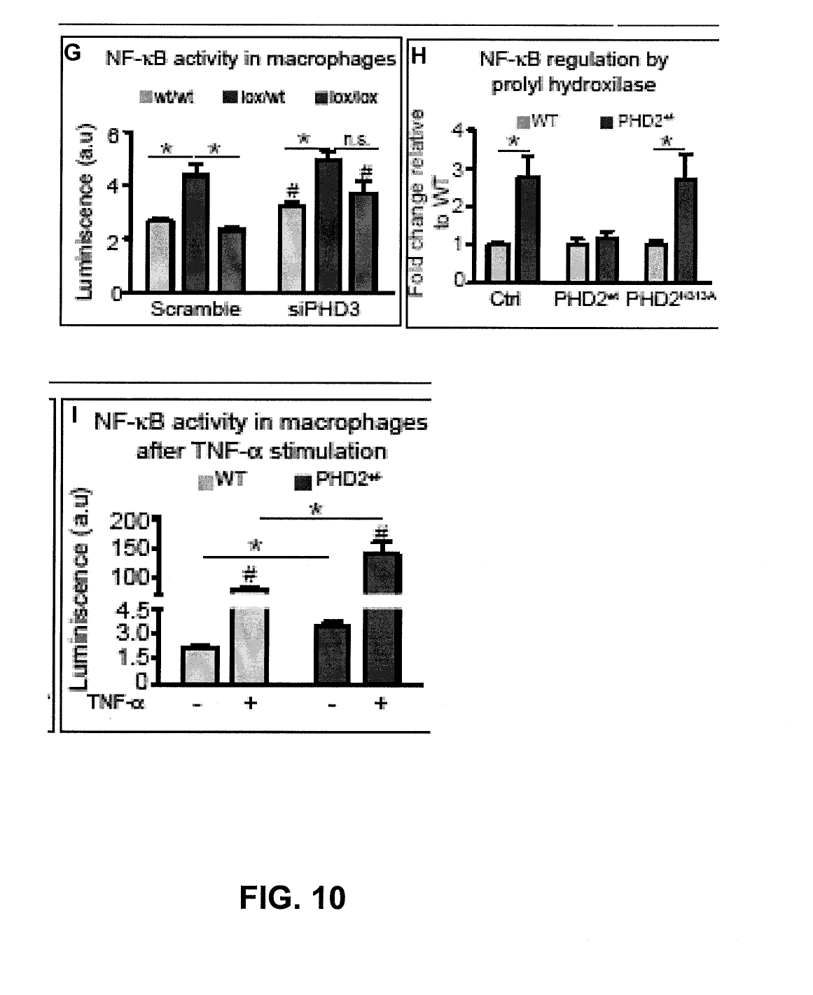

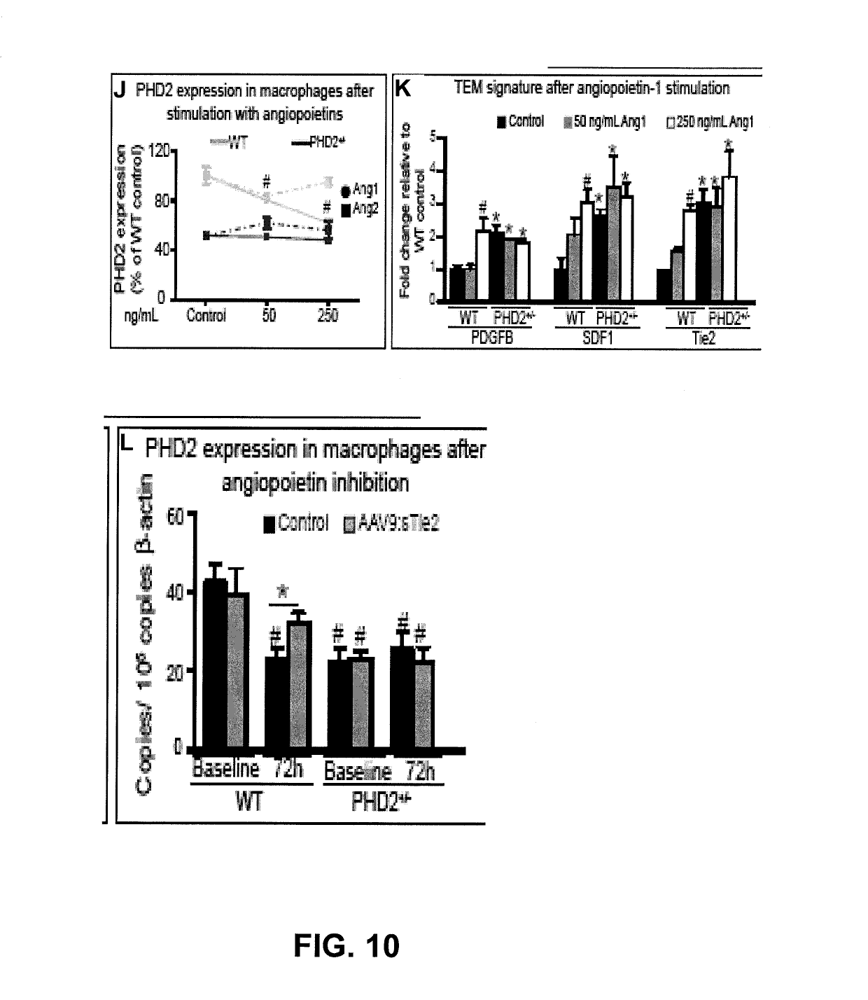

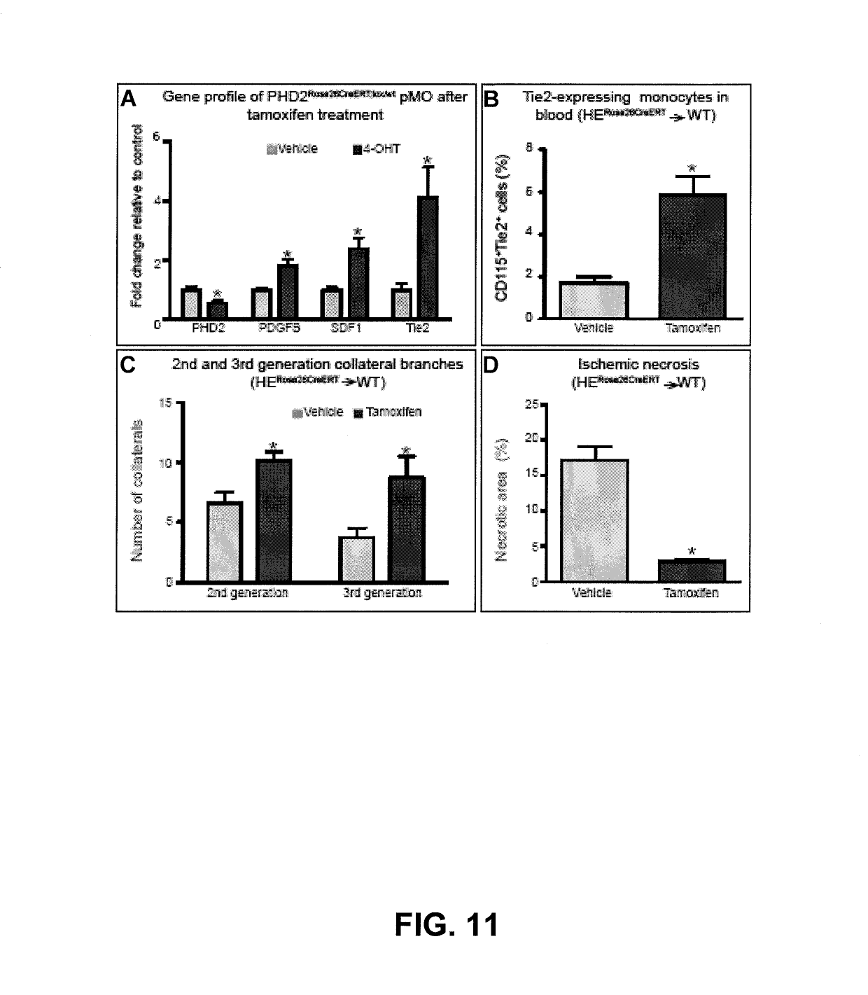

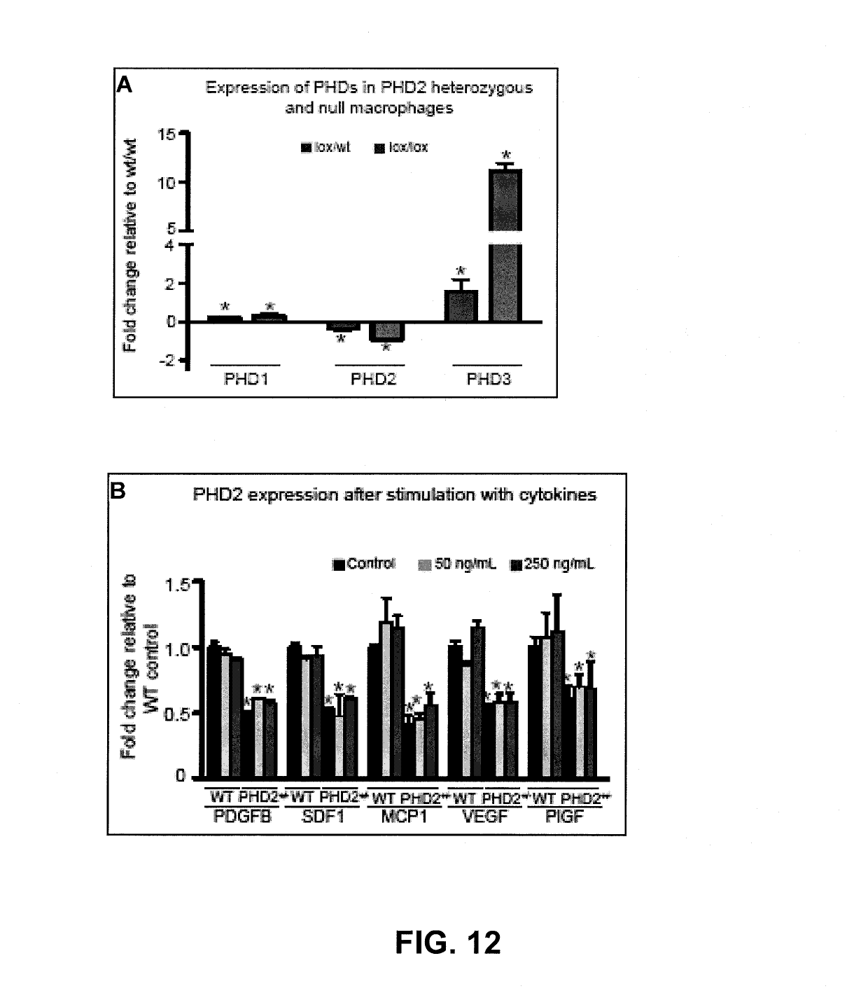

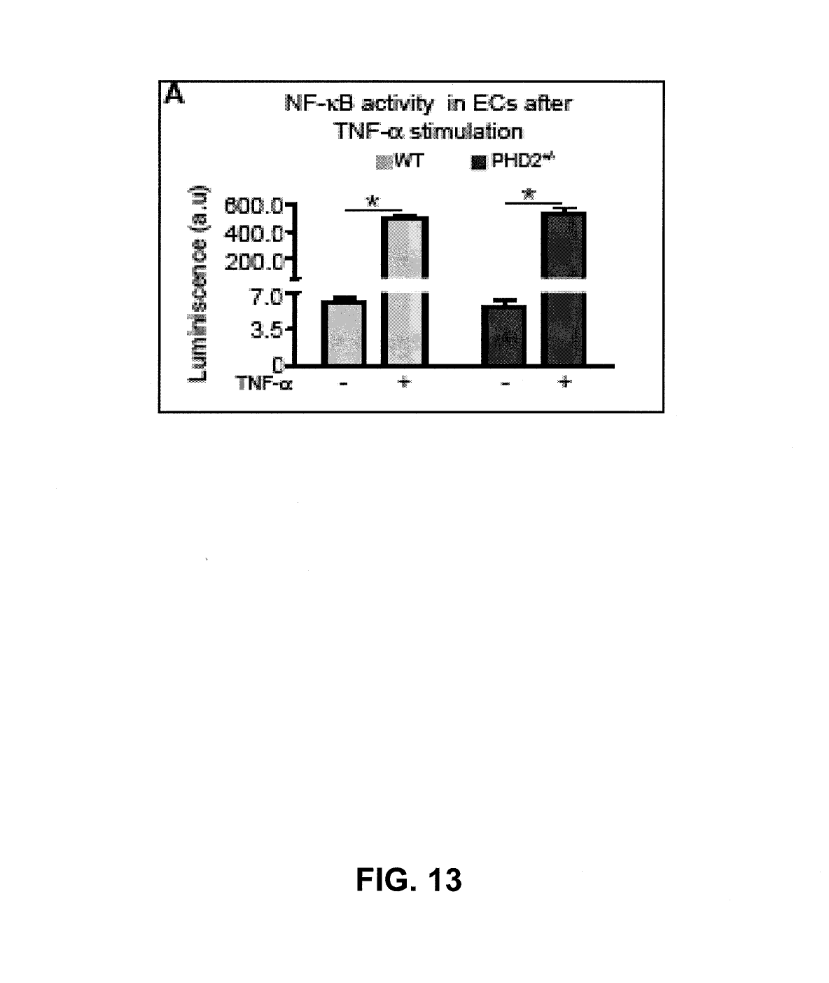

We recently showed that stromal haplodeficiency of PHD2 increases tumor perfusion (Mazzone et al., 2009, see above). Prompted by these results, we examined whether partial loss of PHD2 also enhances perfusion of ischemic tissues. We, therefore, subjected mice to femoral artery ligation, an established procedure that reduces perfusion of the lower limb and causes ischemia in calf i.e., i.e., crural muscle. Laser-doppler measurements revealed that perfusion of the lower hind limb was higher in PHD2^{+/-} than wild-type (WT) mice at 12, 24 and 48 hours after femoral artery ligation, during the critical period when myofibers die if they do not receive sufficient oxygen (FIG. 1, Panel A). The increased perfusion in PHD2^{+/-} mice translated in enhanced physical endurance in ischemic conditions (twelve hours post-ligation), whereas both genotypes exhibited similar running capacity at baseline (FIG. 1, Panel B). Quantification of oxygen levels in the calf by MRI-based oxymetry at twelve hours after ligation revealed that femoral ligation induced a drop of oxygen tension by 66% in WT and 46% in PHD2^{+/-} mice (FIG. 1, Panel C). Such differences in oxygen tension have been shown to influence the outcome of the ischemic disease.²⁶ Staining for the hypoxia-marker pimonidazole showed that the hypoxic area in the crural muscle of ligated limbs was 37.1±3.0% in WT mice but only 16.0±7.0% in PHD2^{+/-} mice (FIG. 1, Panel D, and data not shown). Pimonidazole staining of baseline WT and PHD2^{+/-} crural muscles was negative (data not shown). In accordance with findings that oxygen consumption in conditions of low oxygen availability is associated with formation of reactive oxygen species (ROS), after twelve hours of ischemia, WT but not PHD2^{+/-} crural muscles stained strongly for 8-hydroxy-2-deoxyguanosine (8-OHdG), a marker of deoxyguanosine oxidation (FIG. 2, Panel A). At baseline, oxidative stress in the crural muscle was comparable in both genotypes (data not shown). We next determined whether the decreased drop in perfusion and thus oxygen tension in PHD2^{+/-}-ligated limbs prevented ischemic necrosis. Histological analysis of the crural muscles, i.e., soleus, showed extensive ischemic damage in WT mice at 72 hours after ischemia (data not shown). In PHD2^{+/-} mice, ischemic necrosis of the soleus was reduced by more than 50% (FIG. 1, Panel E). In accordance, crural muscle viability after ischemia was almost double in PHD2^{+/-} than in WT mice (FIG. 1, Panel F). Compared to WT mice, muscle fibers in PHD2^{+/-} mice also showed fewer signs of regeneration as assessed by BrdU staining, confirming that they were less damaged (FIG. 2, Panel B, and data not shown).

Upon femoral artery ligation, growth factors released by the ischemic crural muscle promote angiogenesis. Indeed, in WT mice, 14 days after femoral artery occlusion, vessel density and total vessel area in near-completely regenerated regions of the soleus (an oxidative unit of the crural muscle) were increased, respectively, by 33% and 70% (FIG. 1, Panel D, and data not shown). In contrast, in PHD2^{+/-} mice, these parameters remained unchanged compared to the baseline, likely because these muscles never experienced sufficient ischemia to stimulate angiogenesis (FIG. 2, Panels C and D).

We also wanted to assess whether PHD2^{+/-} mice were protected against myocardial ischemia and, therefore, performed ligation of the left anterior descending coronary artery of WT and PHD2^{+/-} hearts. The infarcted area was measured in desmin-stained cross-sections 24 hours after coronary ligation. Desmin-negative area in the left ventricle was about 60% in WT hearts while 40% in PHD2^{+/-} hearts (FIG. 1, Panel G, and data not shown). Compared to WT hearts, gelatin-bismuth angiographies revealed higher perfusion of PHD2^{+/-} infarcted hearts (FIG. 1, Panels H and I, and data not shown). Similar features of increased bismuth⁺ vessel area and density were observed in the non-infarcted region of PHD2^{+/-} hearts (FIG. 1, Panels H and I).

Thus, PHD2 haplodeficiency greatly preserves perfusion and reduces tissue damage in ischemia.

### PHD2 Haplodeficiency Elicit s “Collateral Vessel Preconditioning”

To assess how heterozygous deficiency of PHD2 prevents tissue ischemia, further experiments were perfoinied. Since PHD2^{+/-} muscles were protected against ischemic damage already twelve hours after femoral artery ligation, we hypothesized that PHD2 haplodeficient mice were preadapted to and, therefore, capable to better tolerate the ischemic insult. The number and caliber of preexisting collaterals (primary, secondary and tertiary branches) are major determinants of the severity of tissue injury in occlusive diseases since these conduits allow blood flow to bypass the obstruction.^{2, 4, 5} We, therefore, investigated whether PHD2^{+/-} mice showed improved collateral growth at baseline, independently of ischemia. Macroscopic counting of collateral arteries on gelatin-bismuth angiographies in the thigh of non-occluded limbs revealed a similar number of primary branches in both genotypes. PHD2^{+/-} mice, however, had 1.7- and two-fold more secondary and tertiary collateral arteries, respectively (FIG. 3, Panels A and B). Histological analysis of the adductor muscles (located in the inner thigh, where collaterals form) showed that the total area and density of bismuth-positive collaterals at baseline were, respectively, 2.0- and 2.3-fold higher in PHD2^{+/-} than WT mice FIG. 3, Panels C and D, and data not shown). Not only the first generation collaterals are better perfused, but there is also an increase in the number of functional second and third generation collaterals. As this is an increase in the number of perfused vessels, but not in the number of vessels per se, the difference is due to increased maturation (widening) of existing vessels. In other words, the collaterals are more stable and allow better perfusion.

Conversely, capillary density and area in the adductor were comparable in both genotypes (FIG. 4, Panels A and B). Consistent with these results, X-ray radiography (FIG. 3, Panels E and F) and micro-CT scans showed a higher number of large vessels (>240 μm in diameter) in PHD2^{+/-} than WT thighs at baseline (FIG. 3, Panel G), whereas smaller vessels (<240 μm in diameter) were not changed (FIG. 4, Panel C). Similar results were obtained in PHD2^{+/-} hearts at baseline, which displayed a higher density of large vessels (FIG. 3, Panels H-J) and a similar number of small vessels and capillaries when compared to controls (FIG. 4, Panels D and E). A distinction was made between vessels with a cross-section surface larger than 240 μm² and those with a smaller cross-section (for methodology, see Luttun et al., 2002). This allows distinguishing between functionally active vessels (>240 μm²) and vessels that are too small to allow considerable blood flow (<240 μm²).

After femoral artery ligation, evaluation of gelatin-bismuth angiographies in WT limbs showed a 30% induction of the collateral vascularization at 12 and 72 hours post-ischemia (FIG. 3, Panels A and B). Conversely, in PHD2^{+/-} mice, the number of collaterals in the adductor did not significantly change after occlusion, likely because they were already expanded at baseline (FIG. 3, Panels A and B). Nevertheless, there were still more secondary and tertiary collateral branch arteries after ischemia in PHD2^{+/-} than WT mice (FIG. 3, Panels A and B). Histological analysis confirmed that at 12 and 72 hours post-ligation, the bismuth-positive collateral area and density in adductor muscles were still higher in PHD2^{+/-} than WT controls (FIG. 3, Panels C and D).

To increase blood flow, collateral vessels undergo extensive remodeling (arteriogenesis) and thus the tunica media, consisting of α-smooth muscle actin (αSMA)-positive contractile SMCs, becomes thicker and the diameter of the conduit enlarges. Staining of adductor tissue sections for αSMA revealed that number and total area of αSMA collateral vessels were almost double in PHD2^{+/-} muscles at both baseline and after ischemia (FIG. 3, Panels K and L). However, the mean area of αSMA collaterals was higher in PHD2^{+/-} than WT mice only at baseline conditions, since, 72 hours post-ligation, WT collaterals enlarged to the same size as PHD2^{+/-} collaterals (FIG. 3, Panel M, and data not shown). A similar trend was observed by measuring the thickness of the tunica media (FIG. 3, Panel N). These data show that, at baseline conditions, the collateral vessels of PHD2^{+/-} mice were similar to those of WT mice after femoral artery ligation. This “collateral vessel preconditioning” offered PHD2^{+/-} mice a remarkable protection against lethal muscle ischemia.

This experiment shows that PHD2 haplodeficiency indeed promotes collateral vessel remodeling by increased maturation. Also, it shows that PHD2 inhibition before an ischemic event or within the first 72 hours may be particularly useful, e.g., in the case of ischemia-reperfusion injury. Indeed, ischemia is a common problem in surgery, thus, prevention of ischemia by prior inhibition of PHD2 can certainly be envisaged.

### PHD2^{+/-} Macrophages Display a Specific Phenotype

Since inflammatory cells and, in particular, macrophages are known to produce SMC/EC-mitogens, cytokines and proteases during collateral growth, it was hypothesized that the increased collateralization in PHD2^{+/-} muscles was due to enhanced infiltration of leukocytes in response to HIF-mediated release of chemoattractant proteins. Surprisingly, when we measured the density of leukocytes and macrophages by staining adductor tissue sections for CD45 and F4/80, respectively, there was no difference between both genotypes at baseline (FIG. 5, Panels A and B). Ligation of the femoral artery induced a significant but comparable increase in inflammatory cell accumulation in WT and PHD2^{+/-} adductors. Consistently, RNA levels of MCP1, one of the most important proinflammatory cytokines in limb ischemia, did not differ in the two genotypes, either at baseline or after femoral artery occlusion (FIG. 6, Panel A).

We, therefore, explored whether the phenotype, not the quantity, of the infiltrating macrophages was different in PHD2^{+/-} and WT mice. We measured the density of wound-healing/proangiogenic macrophages, which can be identified by their enhanced expression of the mannose receptor, MRC1/CD206 (Pucci et al., 2009, see above; De Palma et al., Cancer Cell 8:211-26, 2005), and correspond to M2-polarized macrophages (Mantovani and Sica, Curr. Opin. Immunol. 22:231-7; Mantovani et al., Arterioscler. Thromb. Vasc. Biol. 29:1419-23, 2009). Notably, co-staining adductor sections for MRC1 and the macrophage-specific marker F4/80 revealed that the number of F4/80⁺MRC1⁺ cells was augmented by 75% at baseline conditions in PHD2^{+/-} as compared to WT mice (FIG. 5, Panel C, and data not shown). At 72 hours after ligation, their numbers were increased by 95% in WT mice and by 54% in PHD2^{+/-} mice, but remained higher by 35% in ischemic PHD2^{+/-} than WT mice (FIG. 5, Panel C).

Prompted by these results, we gene-profiled WT and PHD2^{+/-} macrophages collected by peritoneal lavages (peritoneal macrophages, pMΦ) and analyzed the expression level of proangiogenic/proarteriogenic, proinflammatory and antiangiogenic genes. Remarkably, the genes that were up-regulated in PHD2^{+/-} macrophages were markers of wound-healing/proangiogenic (i.e., M2-like) macrophages, and included Tie2, Arg1, CXCR4, Nrp1, HGF, MMP2, FIZZ, CXCL12/SDF1, PDGFB and TGFβ (FIG. 5, Panel D). Of note, these molecules have been reported to play an important role during the arteriogenic process (Schaper, Basic Res. Cardiol. 104:5-21, 2009). Interestingly, several proinflammatory or antiangiogenic (i.e., Th1/M1 -type) molecules were down-regulated in PHD2^{+/-} macrophages; these included IL1β, IL6, NOS2, and IL12 (FIG. 5, Panel D). The changes in the molecular signature of macrophages were already detectable at baseline conditions, since F4/80⁺ cells freshly sorted from adductor muscles of PHD2^{+/-} mice, expressed higher levels of PDGFB, SDF1, Tie2, MMP2, Nrp1 (FIG. 5, Panel E). After 72 hours of ischemia, the expression levels of these markers, except SDF1, caught-up in WT tissue macrophages (FIG. 5, Panel E). All these genes and others were similarly expressed in WT and PHD2^{+/-} ECs, freshly isolated from adductor muscles at baseline and in ischemia (Table 1). Thus, PHD2^{+/-} macrophages display a unique and cell-specific gene signature, which is reminiscent, at least in part, of that of M2-polarized macrophages.

TABLE 1
GENE EXPRESSION IN WT AND
PHD2^{+/-} ENDOTHELIAL CELLS.

| | WT | | PHD2^{+/-} | |
|---|---|---|---|---|
| GENE | BASELINE | LIGATED | BASELINE | LIGATED |
| Phd2 | 1 ± 0.16 | 1.2 ± 0.17 | 0.5 ± 0.03 * | 0.5 ± 0.09 * |
| Tie2 | 1 ± 0.02 | 1.1 ± 0.39 | 1.1 ± 0.13 | 0.9 ± 0.40 |
| Ang1 | 1 ± 0.25 | 1 ± 0.5 | 1.25 ± 0.5 | 1.25 ± 0.75 |
| Ang2 | 1 ± 0.19 | 1.5 ± 0.05 ^{#} | 1.04 ± 0.09 | 1.5 ± 0.16 ^{#} |
| Cxcl1 | 1 ± 0.38 | 2.5 ± 0.71 | 1.3 ± 0.29 | 2.8 ± 0.80 |
| Cxcl2 | n.d. | | n.d. | |
| Cxcr4 | 1 ± 0.11 | 0.6 ± 0.11 ^{#} | 0.9 ± 0.17 | 0.5 ± 0.08 ^{#} |
| Cx3cr1 | 1 ± 0.13 | 0.6 ± 0.13 | 0.9 ± 0.13 | 0.6 ± 0.11 |
| hgf | 1 ± 0.01 | 1.2 ± 0.36 | 1.2 ± 0.11 | 1.0 ± 0.25 |
| Pdgfb | 1 ± 0.06 | 0.9 ± 0.08 | 0.9 ± 0.15 | 0.9 ± 0.19 |
| Plgf | 1 ± 0.16 | 3.1 ± 0.84 | 1.5 ± 0.38 | 3.3 ± 0.56 |
| Rantes | n.d. | | n.d. | |
| Ccl17 | n.d. | | n.d. | |
| Ccl22 | n.d. | | n.d. | |
| Flk1 | 1 ± 0.14 | 0.4 ± 0.09 ^{#} | 0.86 ± 0.09 | 0.4 ± 0.04 ^{#} |
| Flt1 | 1 ± 0.26 | 0.7 ± 0.17 | 0.9 ± 0.47 | 0.7 ± 0.24 |
| sFlt1 | 1 ± 0.10 | 0.7 ± 0.03 | 1.0 ± 0.10 | 0.7 ± 0.12 |
| Nrp1 | 1 ± 0.04 | 0.4 ± 0.08 ^{#} | 0.9 ± 0.38 | 0.5 ± 0.05 ^{#} |
| Cdh5 | 1 ± 0.06 | 0.8 ± 0.06 | 1.2 ± 0.17 | 0.8 ± 0.17 |
| Vegfa | 1 ± 0.5 | 1 ± 0.30 | 0.8 ± 0.20 | 1.2 ± 0.20 |
| Mmp2 | 1 ± 0.30 | 1.5 ± 0.42 | 1.2 ± 0.37 | 1.4 ± 0.36 |
| Mmp9 | 1 ± 0.48 | 0.6 ± 0.26 | 1.2 ± 0.68 | 0.5 ± 0.11 |
| Sdf1 | 1 ± 0.12 | 1.2 ± 0.26 | 0.8 ± 0.12 | 1.0 ± 0.22 |
| Tgfβ | 1 ± 0.04 | 0.8 ± 0.05 | 0.9 ± 0.09 | 1.0 ± 0.38 |
| IL1β | n.d. | | n.d. | |
| IL6 | 1 ± 0.23 | 1.6 ± 0.28 | 1.1 ± 0.14 | 1.72 ± 0.31 |
| Nos3 | 1 ± 0.17 | 0.8 ± 0.11 | 0.8 ± 0.16 | 0.8 ± 0.14 |
| Mcp1 | 1 ± 0.18 | 5.4 ± 0.13 ^{#} | 0.9 ± 0.16 | 6.0 ± 0.13 ^{#} |
| Tnfα | 1 ± 0.21 | 0.8 ± 0.28 | 1.0 ± 0.18 | 0.7 ± 0.15 |
| Cxcl10 | 1 ± 0.23 | 2.1 ± 0.19 ^{#} | 1.1 ± 0.41 | 2.4 ± 0.27 ^{#} |
| IL12 | n.d. | | n.d. | |
| IFNβ | n.d. | | n.d. | |
| Cox2 | 1 ± 0.12 | 1.2 ± 0.04 | 1.1 ± 0.2 | 1.1 ± 0.2 |
| Jagged1 | 1 ± 0.09 | 0.9 ± 0.07 | 1.1 ± 0.10 | 1.0 ± 0.15 |
| Icam1 | 1 ± 0.11 | 0.8 ± 0.13 | 0.8 ± 0.09 | 0.6 ± 0.08 |

The data represent the expression analysis of endothelial cells, freshly sorted from WT and PHD2^{+/-} adductor muscles at baseline and 72 hours after femoral artery occlusion (N=4-8, P<0.05). Data are normalized toward the expression levels of WT ECs at baseline; n.d.=not determined. Asterisks denote statistical significance versus WT. Hash signs denote statistical significance compared to the baseline.

### Heterozygous Deficiency of PHD2 in Myeloid Cells Prevents Ischemic Damage

To investigate whether reduced levels of macrophage-derived PHD2 displays collateral vessel preconditioning and thus protection against ischemia, we generated conditional PHD2-deficient mice lacking one or two PHD2 alleles specifically in myeloid cells (PHD2^{LysCre;lox/wt} and PHD2^{LysCre;lox/lox}, respectively) by intercrossing PHD2^{lox/w} and PHD2^{lox/lox} mice with LysM:Cre mice expressing the Cre-recombinase under the control of the myeloid-specific lysozyme M promoter. In contrast to PHD2 knock-out mice, which die between E12.5 and E14.5 due to placental defects, mice with homozygous deficiency of PHD2 in myeloid cells (PHD2^{LysCre;lox/lox}) are viable and fertile. Gelatin-bismuth angiographies revealed a higher number of secondary and tertiary collateral branch arteries in heterozygous PHD2^{LysCre;lox/wt} mice while arterialization was unchanged in PHD2^{LysCre;lox/lox} mice (FIG. 7, Panels A and B). Histological analysis of the same adductor samples showed that the total area and density of bismuth-positive collaterals were higher in PHD2^{LysCre;lox/wt} but not in PHD2^{LysCre;lox/lox} mice compared to control mice (FIG. 7, Panels C and D). Collateral vessel preconditioning conferred ischemic protection since, 72 hours after femoral artery occlusion, muscle necrosis was reduced by 67% in PHD2^{LysCre;lox/wt} but not in PHD2^{LysCre;lox/lox} mice (FIG. 7, Panel E, and data not shown). Similarly, in ischemia, the running capacity of PHD2^{LysCre;lox/wt} but not of PHD2^{LysCre;lox/lox} mice was 1.6-fold higher compared to PHD2^{LysCre;wt/wt} mice while comparable at baseline (FIG. 7, Panel F).

To further explore whether the increased arteriogenesis in PHD2 haplodeficient mice could be attributed to the lack of one PHD2 allele in macrophages, we transplanted WT or PHD2^{+/-} (hereafter HE) bone marrow of syngenic mice, ubiquitously expressing GFP, into lethally irradiated WT recipients (referred to as WT→WT and HE→WT mice, respectively) or into lethally irradiated PHD2^{+/-} recipients (referred to as WT→HE and HE→HE mice, respectively). Collateral arteries were quantified at five weeks after bone marrow transplantation, when hematopoietic reconstitution with GFP⁺ blood cells was about 82% and differential white blood counts were comparable in all the groups (not shown). Histological analysis of gelatin bismuth-based angiographies revealed greater numbers and area of collateral vessels in HE→WT than WT→WT mice while not differing from HE→HE mice, supporting the key role of bone marrow-derived cells in enhancing collateralization (FIG. 7, Panels G and H). Interestingly, collateral vessel parameters in WT→WT and WT→HE mice were comparable (FIG. 7, Panels G and H), indicating that bone marrow-derived cells are also important to sustain preexisting arteries in PHD2 heterozygous mice. In accordance, ischemic necrosis at 72 hours post-ligation was prevented in HE→HE and HE→WT mice, while it did not reach statistical significance in WT→HE mice (FIG. 7, Panel I). We also assessed whether transplantation of HE bone marrow into lethally irradiated WT recipients would suffice to improve the physical endurance in ischemia. In a treadmill test, the running capacity of HE→WT mice was twice as good as in WT→WT mice at twelve hours after femoral artery ligation while no differences were detected at baseline conditions (FIG. 7, Panel J).

Finally, we generated another strain lacking one PHD2 allele in all hematopoietic and endothelial lineage cells (PHD2^{Tie2Cre;lox/wt}) by using the Tie2Cre deleter mouse line (Mazzone et al., Cell, 2009). Reciprocal bone marrow transplantation of PHD2^{Tie2Cre;lox/wt} and PHD2^{Tie2Cre;wt/wt} mice revealed that increased arteriogenesis of PHD2 heterozygous mice was specifically caused by loss of one PHD2 allele in bone marrow-derived inflammatory cells but not in endothelial cells (Table 2). Reduction of collateral branches in PHD2^{Tie2Cre;lox/wt} recipient mice transplanted with a WT bone marrow (PHD2^{Tie2Cre;wt/wt}), further support the idea that inflammatory cells are required for artery maintenance in PHD2 haplodeficient mice (Table 2). Deletion of a PHD2 allele specifically in ECs or SMCs did not affect arteriogenesis (Table 3).

Thus, lower levels of PHD2 in bone marrow-derived myeloid cells, but not in ECs and/or SMCs, increase collateral vessel formation and prevent ischemic damage.

TABLE 2
COLLATERALIZATION IN MICE HAPLODEFICIENT FOR PHD2 IN
THE HEMATOPOIETIC AND/OR ENDOTHELIAL LINEAGE CELLS.

| | Donor | | | |
|---|---|---|---|---|
| | PHD2^{Tie2Cre; wt/wt} | PHD2^{Tie2Cre; lox/wt} | PHD2^{Tie2Cre; wt/wt} | PHD2^{Tie2Cre; lox/wt} |
| Recipient | PHD2^{Tie2Cre; wt/wt} | PHD2^{Tie2Cre; wt/wt} | PHD2^{Tie2Cre; lox/wt} | PHD2^{Tie2Cre; lox/wt} |
| 2^{nd} generation | 5.9 ± 1.0 | 12.7 ± 1.4 *^{#} | 8.2 ± 0.7 | 14.4 ± 1.6 *^{#} |
| collaterals | | | | |
| 3^{rd} generation | 3.8 ± 0.7 | 7.2 ± 1.3 *^{#} | 2.7 ± 1.1 | 7.1 ± 1.0 *^{#} |
| collaterals | | | | |

Reciprocal bone marrow transplantation in lethally irradiated mice reveals that the enhanced arteriogenesis of PHD2 heterozygous mice is specifically caused by loss of one PHD2 allele in bone marrow-derived cells (third column) but not in endothelial cells (fourth column) compared to WT controls (second column). Combined deletion of one PHD2 allele in both inflammatory cells and ECs (fifth column) does not modify the biological effect elicited on collateral arteries by PHD2 haplodeficient inflammatory cells only. Asterisks denote statistical significance versus PHD2^{Tie2Cre;wt/wt}→PHD2^{Tie2Cre;wt/wt}. Hash signs denote statistical significance compared to PHD2^{Tie2Cre;wt/wt}→PHD2^{Tie2Cre;lox/wt}.

TABLE 3
HETEROZYGOUS DEFICIENCY OF PHD2 IN ENDOTHELIAL
CELLS OR SMOOTH MUSCLE CELLS DOES NOT CONFER
COLLATERAL PRECONDITIONING.

| | PHD2^{Cre; wt/wt} | | PHD2^{Cre; wt/lox} | |
|---|---|---|---|---|
| | 2^{nd} | 3^{rd} | 2^{nd} | 3^{rd} |
| Promoter of Cre | generation | generation | generation | generation |
| recombinase | collaterals | collaterals | collaterals | collaterals |
| VE-Cadherin | 9.0 ± 0.6 | 10.8 ± 1.3 | 9.9 ± 1.2 | 11.0 ± 2.0 |
| PDGFRB | 8.2 ± 0.9 | 7.4 ± 1.4 | 7.2 ± 1.1 | 6.7 ± 1.6 |

The data represent the number of secondary and tertiary collateral branches in mice haplodeficient for PHD2 in ECs or SMCs at baseline. Mice, where a single PHD2 was floxed, were intercrossed with deleters expressing the Cre recombinase under an EC-specific promoter, i.e., VE-Cadherin, or a SMC-specific promoter, i.e., PDGFRB.

### Macrophage-Derived SDF1 and PDGFB Promote Arteriogenesis

In order to unravel the biological mechanism underlying the arteriogenic phenotype, we assessed how WT and PHD2^{+/-} macrophages affect the behavior of ECs and SMCs, the two main cellular components of arteries. First, we evaluated the chemotactic potential of primary ECs and SMCs toward WT and PHD2^{+/-} macrophages. EC migration toward WT or PHD2^{+/-} macrophages was comparable, and fifty times higher than toward culture medium alone (FIG. 8, Panel A, and data not shown). SMCs migrated 6.5 times more efficiently when WT macrophages were seeded in the lower chamber of the transwell (compared to control medium), whereas migration towards PHD2^{+/-} macrophages was 44 times higher (FIG. 8, Panel B, and data not shown). Given the finding that the two cytokines SDF1 and PDGFB were up-regulated the highest in PHD2^{+/-} macrophages (see FIG. 5, Panel D), we tested whether inhibiting these pathways, alone or in combination, would abrogate chemoattraction of SMCs toward PHD2^{+/-} macrophages. Combined inhibition of SDF1 and PDGFB signaling by AMD3100 and imatinib, respectively, abrogated the increased migration of SMCs toward PHD2^{+/-} macrophages, while either treatment alone was not effective (FIG. 8, Panel C). Similarly, when silencing both SDF1 and PDGFB in PHD2^{+/-} macrophages, SMC migration was almost completely prevented, though, by genetic knock-down, each shRNA alone was already partly effective (FIG. 9).

To assess the influence of soluble factors released by WT and PHD2^{+/-} macrophages on EC and SMC growth, we performed a cell viability assay. We seeded ECs and SMCs on the upper side of a 0.4 μm-pore filter (that does not allow cell migration but only protein diffusion), and WT or PHD2^{+/-} macrophages in the lower chamber. Notably, growth of SMCs was enhanced by soluble factors released from PHD2^{+/-} (versus WT) macrophages (FIG. 8, Panel D). EC growth was not differently affected by WT and PHD2^{+/-} macrophages (FIG. 8, Panel E). SMCs display a proliferative (or synthetic) phenotype during the phase of active growth in contrast to the contractile phenotype in mature vessels. The proliferative or synthetic phenotype is characterized by the reduction of contractile proteins including smoothelin, NmMHC, αSMA, and of calponin family proteins, i.e., calponin-1 and Sm22α. The down-modulation of these genes in SMCs indicates that these cells are under the influence of growth factors and are able to migrate and to proliferate. Consistent with the enhanced growth of SMCs seeded in the presence of PHD2^{+/-} macrophages, conditioned medium from PHD2^{+/-} macrophages reduced the expression level of calponin-1, SM22α, smoothelin, NmMHC and αSMA, therefore, supporting a proliferative phenotype (FIG. 8, Panels F-J). Unlike what we observed in the migration assays, AMD3100 or imatinib alone abrogated the increased SMC growth by PHD2^{+/-} macrophages. The combination of both AMD3100 and imatinib did not elicit an additive effect (FIG. 8, Panel K). Similarly, either single or double knock-down of SDF1 and PDGFB in PHD2^{+/-} macrophages hindered SMC growth (data not shown).

Prompted by the in vitro results, we treated WT→WT and HE→WT mice with daily administration of AMD3100 (5 mg/kg) or imatinib (50 mg/kg), alone or in combination. In vivo, each drug alone only partially prevented the increased foiination of second generation collateral branches in HE→WT mice (FIG. 8, Panel L), while third generation collaterals were affected by either treatment alone (FIG. 8, Panel M). However, the combination of AMD3100 and imatinib more potently prevented collateralization in the adductor of these mice. In WT→WT mice, the number of collateral branch arteries was not affected in all conditions tested (FIG. 8, Panels L and M). Thus, in mice with reduced levels of myeloid PHD2, combined PDGFB and SDF1 pathway activation is necessary to complete the arteriogenic process.

### The Macrophages That Promote Arteriogenesis in PHD2^{+/-} Mice Are Reminiscent of TEMs

Tie2 is a gene recently found to be significantly up-regulated in a subpopulation of macrophages, known as TEMs, which express a M2-like, wound healing/proangiogenic phenotype (Pucci et al., 2009; De Palma et al., 2005). Since Tie2 was strongly induced in PHD2^{+/-} macrophages, we explored if this increase was due to an enhanced fraction of TEMs in the total macrophage population. As tumor TEMs express MRC1 to higher levels than classically activated macrophages/inflammatory macrophages (Pucci et al., 2009), and because we found that PHD2^{+/-} adductors display enhanced infiltration of F4/80⁺MRC1⁺ macrophages (FIG. 5, Panel C), we stained adductor sections from WT and PHD2^{+/-} mice for F4/80, MRC1 and Tie2 in order to rigorously identify TEMs. At baseline conditions, F4/80⁺MRC1⁺Tie2⁺ TEMs were scarce in WT mice but were four times more abundant in PHD2^{+/-} mice (FIG. 10, Panel A). Seventy-two hours after femoral artery occlusion, the density of TEMs was 3.2 times higher in WT but 1.3-fold increased in PHD2^{+/-} mice toward the baseline (FIG. 10, Panel A). Thus, TEM density was still 1.6-fold higher in ischemic PHD2^{+/-} than WT mice (FIG. 10, Panel A). The increased presence of tissue-resident TEMs in PHD2^{+/-} than WT mice was not due to a differential expression of the Tie2 ligands, angiopoietin-1 and angiopoietin-2, since transcript levels of these two cytokines were similar in WT and PHD2^{+/-} adductors at baseline and ischemic conditions (FIG. 6, Panels B and C). When we measured Tie2-expressing monocytes (gated as CD115⁺Tie2⁺ leukocytes) in the blood, we found a 3.4-fold higher TEM frequency in PHD2^{+/-} than WT mice at baseline conditions (FIG. 10, Panel B). Interestingly, 72 hours after femoral artery ligation, the frequency of circulating TEMs was reduced by 3.4-fold in WT and 2.2-fold in PHD2^{+/-} mice, although this decrease reached statistical significance in WT mice only (FIG. 10, Panel B). Similar results were observed when quantifying the transcript levels of Tie2 in WT and PHD2^{+/-} CD115⁺ circulating monocytes, although the overall expression of Tie2 was low (FIG. 10, Panel C). In F4/80⁺ tissue macrophages Tie2 expression was almost 100 times higher than in monocytes in general. After ligation, Tie2 transcript levels were further augmented but only in WT macrophages, likely because PHD2^{+/-} macrophages presented higher Tie2 expression already at baseline (FIG. 10, Panel C). In mice, expression of Gr1 distinguishes “inflammatory” monocytes (CD115⁺Gr1^{high}) from “resident” monocytes (CD115⁺Gr1^{low}).^{40, 41} Circulating TEMs in PHD2^{+/-} mice are mostly CD115⁺Gr1^{low} (data not shown). These data suggest that, in ischemia, TEMs are recruited from the blood to the adductor where they trigger the arteriogenic process.

To address if TEMs are functionally involved in the maturation of collateral arteries and, thus, preadaptation to ischemia in PHD2^{+/-} mice, we used a “suicide” gene strategy based on the Herpes simplex virus thymidine kinase (tk)-ganciclovir (GCV) system (De Palma et al., Nat. Med. 9:789-95, 2003). We transplanted mice with WT or PHD2^{+/-} bone marrow-derived lineage-negative cells transduced with a lentiviral vector (LV) expressing the tk cDNA under the control of the Tie2 promoter/enhancer (Tie2:tk-BMT mice). We also co-transduced WT and PHD2^{+/-} bone marrow cells with a lentiviral vector ubiquitously expressing GFP from the PGK promoter in order to measure bone marrow engraftment in the transplanted mice by scoring GFP expression. By using flow cytometry of GFP⁺ cells and qRT-PCR analysis of integrated vectors in blood cells, we found that PHD2 haplodeficiency in bone marrow hematopoietic cells did not preclude their full engraftment upon transplantation in irradiated mice (GFP⁺ cells, % of leukocyte population: 92.6±2.5% in WT Tie2:tk-BMT mice and 89.3±5.5% in PHD2^{+/-} Tie2:tk-BMT mice; N=6; P=NS; and Table 4).

TABLE 4
VECTOR COPY NUMBER IN BLOOD CELLS OF WT TIE2: TK-
BMT AND PHD2^{+/-} TIE2: TK-BMT MICE.

| | HSV-tk | HIV-gag | PGK: GFP |
|---|---|---|---|
| WT Tie2: tk-BMT | 8.93 ± 0.40 | 15.79 ± 0.02 | 6.85 ± 0.43 |
| PHD2^{+/-} Tie2: tk-BMT | 10.53 ± 0.30 | 19.39 ± 0.01 | 8.85 ± 0.31 |

The data represent the number of integrated LV copies per cell genome (vector copy number, VCN±SEM) of HSV-tk and HIV-gag in blood cells, collected at four weeks after transplantation from WT Tie2:tk-BMT and PHD2^{+/-} Tie2:tk-BMT mice. The VCN of PGK:GFP was obtained by subtracting the VCN of HSV-tk from the total HIV-gag VCN (N=6; P=NS). See Experimental Methods for technical details.

By this approach, bone marrow-derived TEMs can be specifically eliminated upon GCV administration in the transplanted mice. Four weeks after transplantation, WT and PHD2^{+/-} Tie2:tk-BMT mice were treated with either saline or GCV (50 mg/kg daily) for ten days before and three days after femoral artery ligation. The deletion of TEMs was assessed by F4/80 and Tie2 double staining of baseline and ligated adductor sections. We found that GCV treatment reduced the density of F4/80⁺Tie2⁺ cells by 46±10% in WT Tie2:tk-BMT mice and 58±11% in PHD2^{+/-} Tie2:tk-BMT mice at baseline (N=6; P<0.001), and by 39±6% in WT Tie2:tk-BMT mice and 68±5% in PHD2^{+/-} Tie2:tk-BMT mice at 72 hours post-ischemia (N=6; P<0.001).

Remarkably, the formation of secondary and tertiary collateral arteries was completely prevented in GCV-treated PHD2^{+/-} Tie2:tk-BMT mice when compared to the untreated group, as shown by macroscopic evaluation of gelatin bismuth-based angiographies at baseline (FIG. 10, Panels E and F). Consistently, treatment of PHD2^{+/-} Tie2:tk-BMT mice with GCV abolished ischemic protection 72 hours after ligation (FIG. 10, Panel D). Thus, Tie2-expressing macrophages fuel arteriogenesis in PHD2^{+/-} mice.

### Acute Deletion of PHD2 Favors TEMs, Arteriogenesis and Ischemia Protection

In order to strengthen the therapeutic value of our findings, we assessed whether acute deletion of PHD2 induced the same TEM phenotype and, thus, arteriogenesis and protection against ischemia as observed in PHD2^{+/-} mice. To this end, we generated tamoxifen-inducible PHD2 haplodeficient mice (PHD2^{Rosa26CreERT;lox/wt}) where the Rosa26 promoter directs the ubiquitous expression of the fusion protein Cre-ERT2. Peritoneal macrophages were treated in vitro with 2 μM 4-hydroxytamoxifen (4-OHT) or vehicle for 48 hours. Acute deletion of PHD2 increased the expression of PDGFB, SDF1, and Tie2, therefore, resembling the phenotype of PHD2^{+/-} macrophages (FIG. 11, Panel A). To address whether acute deletion of PHD2 in macrophage fuels arteriogenesis, the bone marrow of PHD2^{Rosa26CreERT;lox/wt} mice was transplanted into lethally irradiated WT recipient mice (HE^{Rosa26CreERT}→WT). After five weeks, transplanted mice were treated with vehicle or tamoxifen (1 mg/mouse for five days). At 14 days after tamoxifen treatment, circulating TEMs were almost three-fold increased (FIG. 11, Panel B) and both secondary and tertiary collateral branches were respectively 1.6 and 2.3 times more abundant compared to HE^{Rosa26CreERT}→WT mice treated with the vehicle (FIG. 11, Panel C). Consistent with an increased arteriogenesis, ischemic damage in tamoxifen-treated HE^{Rosa26CreERT}→WT mice was greatly reduced (FIG. 11, Panel D). Thus, acute inactivation of PHD2 might represent a preventive medicine for ischemic diseases.

### Heterozygous Deficiency of PHD2 in Macrophages Enhances NF-κB Activity

PHD2 oxygen sensor negatively regulates HIF accumulation and NF-κB activity. When analyzing the accumulation of HIF-1α and HIF-2α by Western blot analysis, we observed that the levels of HIF-1α and HIF-2α in PHD2 haplodeficient macrophages (PHD2^{LysCre;lox/wt}) were comparable to the control (PHD2^{LysCre,wt/wt}). In contrast, HIF-1α and HIF-2α levels in PHD2 null macrophages (PHD2^{LysCre;lox/lox}) were, respectively, four times and two times higher than in control macrophages (PHD2^{LysCre;wt/wt}; data not shown). We, therefore, quantified NF-κB activity by transducing PHD2^{LysCre;lox/wt}, PHD2^{LysCre;lox/lox}, PHD2^{LysCre,wt/wt} macrophages with a lentiviral vector carrying an NF-κB-responsive firefly luciferase reporter (FIG. 10, Panel G). Interestingly, NF-κB activity was increased by 65% in PHD2 haplodeficient macrophages but unaffected in PHD2 null macrophages.

We hypothesized that other PHD oxygen sensors might compensate for the complete loss of PHD2. We, therefore, measured RNA levels of PHD1, PHD2 and PHD3 in PHD2^{LysCre;wt/wt}, PHD2^{LysCre;lox/wt} and PHD2^{LysCre;lox/lox} macrophages. While PHD2 levels were decreased by 40% and 93% in PHD2^{LysCre;lox/wt} and PHD2^{LysCre;lox/lox} macrophages, respectively, PHD1 and PHD3 transcript levels were 0.2- and 1.5-fold higher in PHD2 haplodeficient macrophages, and 0.3- and 11.2-fold higher in PHD2 null macrophages (FIG. 12, Panel A). PHD3 silencing induced NF-κB activity by 22% and 14% in PHD2^{LysCre;wt/wt} and PHD2^{LysCre;lox/wt} macrophages but by 70% in PHD2^{LysCre;lox/lox} macrophages compared to their scramble controls (FIG. 10, Panel G). To address whether the induction of PHD3 rescued the activation of NF-κB pathway by loss of PHD2, we silenced PHD3 in PHD2^{LysCre;wt/wt}, PHD2^{LysCre;lox/wt} and PHD2LysCre;lox/lox macrophages carrying the NF-κB-responsive luciferase reporter. The knockdown of PHD3 was of 63±0.03%, 60±0.04% and 37±0.01% in PHD2^{LysCre;wt/wt}, PHD2^{LysCre;lox/wt} and PHD2^{LysCre;lox/lox} macrophages compared to their scramble controls (N=4; P<0.001).

These data indicate that PHD3 induction in PHD2 null macrophages is responsible for the repression of NF-κB activity. This may explain, at least in part, the absence of enhanced collateral growth and ischemic protection in mice lacking two PHD2 alleles in myeloid cells. Note that this does not apply to acute deletion of two alleles of PHD2 in myeloid cells; in this case, PHD3 levels will not be up-regulated beforehand. Thus, it is envisaged that acute complete deletion (or complete inhibition) of PHD2 still results in proarteriogenic myeloid cells.

To understand if hydroxylase function was necessary for PHD2-mediated NF-κB regulation, PHD2^{+/-} macrophages were electroporated with a plasmid carrying a wild-type PHD2 (PHD2^{wt}), a hydroxylase-deficient PHD2 containing a mutation at a critical residue in the catalytic site (PHD2^{H313A}) (Jokilehto et al., Exp. Cell Res. 316(7):1169-78 (2010)) or an empty vector as control. Ectopic expression of PHD2^{wt} greatly blunted the activity of NF-κB luciferase induced by PHD2 haplodeficiency, whereas PHD2^{H313A} had no effect (FIG. 10, Panel H), suggesting a functional role for PHD2 hydroxylase activity in the down-regulation of NF-κB pathway. We also assessed the effect of TNF-α, archetypal cytokine activating the canonical NF-κB pathway, in WT and PHD2^{+/-} macrophages and found that TNF-α-induced NF-κB activation was significantly stronger in PHD2 haplodeficient macrophages (FIG. 10, Panel I). In contrast, basal and TNF-α-induced NF-κB activity were comparable in WT and PHD2^{+/-} ECs (FIG. 13). Consistent with an activation of canonical NF-κB pathway, p65 (RelA) and p50 (NF-κB1) protein accumulation was more prominent in PHD2^{+/-} than WT macrophages (data not shown).

To evaluate the involvement of the canonical NF-κB signaling in macrophage skewing by PHD2 haplodeficiency, we generated a myeloid-specific double transgenic strain, heterozygous deficient for PHD2 and null for IKKβ, a positive regulator of NF-κB canonical pathway. Disruption of NF-κB canonical pathway via genetic deletion of IKKβ prevented the up-regulation of Tie2, PDGFB and SDF1 in cultured PHD2 haplodeficient macrophages and abolished the induction of circulating TEMs, the increase of collateral branches and the protection against ischemic necrosis in PHD2 haplodeficient mice.

### PHD2 Levels Are Down-Regulated by Angiopoietins in Monocytes/Macrophages

In ischemia, arteriogenesis takes place in a non-hypoxic environment (Ito et al., 1997; Gray et al., 2007). We, therefore, questioned whether some other mechanisms besides low oxygen tension could lead to down-regulation of PHD2 in a way that resembles heterozygous deficiency of PHD2. Since angiopoietin-1 was previously reported to inhibit the transcription of PHD2 (Chen and Stinnett, Diabetes 57:3335-43, 2008; McMahon et al., J. Biol. Chem. 281:24171-81, 2006), we hypothesized that increased expression of Tie2 ligands by the adductor after femoral artery occlusion (FIG. 6, Panels B and C), would induce Tie2 expression on monocytes via PHD2 down-modulation in a positive feedback loop. Thus, we assessed the expression levels of PHD2 in WT and PHD2^{+/-} primary bone marrow-derived monocyte/macrophage cultures upon stimulation with increasing concentrations of angiopoietin-1 and angiopoietin-2. In WT monocytes/macrophages, angiopoietin-1 down-regulated PHD2 levels in a dose-dependent fashion up to 50% of the basal levels, while angiopoietin-2 induced a 15% reduction only when used at 50 ng/ml (FIG. 10, Panel J). This effect was specific for angiopoietin-1 since other ischemia-induced cytokines such as MCP1, VEGF, PlGF, PDGFB and SDF1 did not affect PHD2 transcripts at any of the concentrations tested (FIG. 12, Panel B). PHD2^{+/-} bone marrow-derived monocyte/macrophage cultures expressed about 50% of the PHD2 levels measured in WT unstimulated controls. However, this expression was not affected by any of the cytokines tested (FIG. 10, Panel J, and FIG. 12, Panel B). Interestingly, PHD2 down-modulation in monocytes/macrophages by 40% after angiopoietin-1 stimulation correlated with increased expression of Tie2, PDGFB and SDF1 (FIG. 10, Panel K). To assess the role of angiopoietins in the regulation of PHD2 expression, WT recipient mice were reconstituted with the bone marrow from WT and PHD2^{+/-} mice (WT→WT and HE→WT, respectively), and then systemically and locally injected with an AAV codifying the extracellular domain of Tie2 (sTie2), or albumin as control. Ten days after injection, we sorted F4/80⁺ tissue-resident macrophages from adductors, both at baseline and 72 hours post-ligation and, thus, measured the transcript levels of PHD2. Strikingly, after ischemia, the levels of PHD2 were halved in WT macrophages, thus resembling the levels of PHD2 in PHD2^{+/-} macrophages at baseline. Angiopoietin blockade, however, greatly prevented this effect in WT macrophages, whereas it was ineffective in PHD2^{+/-} macrophages. At baseline, PHD2 levels were not changed by sTie2 administration in both WT and PHD2^{+/-} macrophages (FIG. 10, Panel L).

These results suggest that angiopoietin release in ischemic can be, at least in part, responsible for PHD2 repression that would ultimately lead to monocyte/macrophage skewing and, thus, arterial collateral branch formation. In other words, angiopoietin administration can be envisaged as a way of inhibiting PHD2 and obtaining the desired proarteriogenic myeloid cells.

### CONCLUSIONS

Specific macrophage subsets/differentiation states have been implicated in the promotion of angiogenesis during cancer and atherosclerosis progression. However, little is known of the significance of macrophage heterogeneity in arteriogenesis and its implications on ischemic diseases. A role of myeloid PHD2 in oxygen delivery by regulating arteriogenesis is proposed herein. Reduced PHD2 levels in macrophages determine a specific gene signature that fosters the arteriogenic program by inducing recruitment and growth of SMCs. This program relies on a NF-κB-dependent up-regulation of macrophage-derived SDF1 and PDGFB, and the angiopoietin receptor, Tie2. It is shown that the combined effect of PDGFB and SDF-1 is essential to the arteriogenic process.

We show that the phenotype of macrophages induced by reduced levels of myeloid PHD2 not only favors the formation of new collateral branches, but is also important for collateral vessel homeostasis. Under steady-state conditions, blood monocytes act as circulating precursors that migrate into non-inflamed tissue for replacing certain subsets of tissue macrophages and dendritic cells. PHD2 haplodeficient bone marrows in WT recipient mice enhanced collateral formation. However, when PHD2^{+/-} mice were transplanted with a WT bone marrow, preexisting collaterals regressed to the same level as in WT mice, suggesting a role of tissue macrophages in sustaining artery maintenance. The proarteriogenic tissue macrophages identified in the present study are reminiscent of the M2-like, proangiogenic macrophage subset, known as TEMs, which are found in tumors and developing or regenerating tissues (Pucci et al., 2009). The identified proarteriogenic macrophages do not up-regulate either VEGF or inflammatory genes, but express increased levels of Tie2, Nrp1, PDGFB and SDF1. Remodeling tissue- and tumor-resident TEMs appear to originate from a distinct population of circulating Tie2-expressing monocytes (Pucci et al., 2009). This corresponds to our data. Tie2-expressing monocytes as well as Tie2-expressing macrophages were increased, respectively, in the peripheral blood and adductor of PHD2 haplodeficient mice and their depletion prevented the enhanced formation of collateral arteries.

After femoral artery occlusion, the bulk of blood flow is redirected into collateral conduits, thus generating shear stress that induces release of chemoattractant molecules, including angiopoietin-1 and angiopoietin-2. In tumor settings, angiopoietin-2, one of the four known ligands of Tie2, recruits TEMs to the tumor and enhances their proangiogenic activity in the tumor microenvironment (Lewis et al., Cancer Res. 67:8429-32, 2007). However, the present results are the first to describe the involvement of Tie2-expressing monocytes in the arteriogenic process.

Collateral formation is a hypoxia-independent process. Thus, can PHD2 be inactivated in an oxygen-independent manner? Besides hypoxia, several cytokines can down-regulate PHD2 expression. We now show that angiopoietins partially down-regulate the expression of PHD2 in mononuclear phagocytes. Besides angiopoietins, other cytokines such as TGFβ might contribute to the repression of PHD2 in ischemia (McMahon et al., 2006). Interestingly, angiopoietins as well as TGFβ have been reported to enhance collateral vascularization, in part, through a direct effect on monocytes. Without being bound to a particular mechanism, the model we propose is as follows. After femoral artery ligation, release of cytokines induces the down-regulation of PHD2 in monocytes. This, in turn, unleashes NF-κB signals that are independent from HIFs and PHD2 enzymatic activity (Chan et al., Cancer Cell 15:527-38, 2009). NF-κB activation will then lead to Tie2 expression on the cell membrane of circulating monocytes. In a positive feedback loop, angiopoietins or other factors released after major artery occlusion, may recruit Tie2⁺ monocytes to the pericollateral region where they will fuel the tissue with SDF1 and PDGFB. The combined activity of these two cytokines will induce SMC migration, positioning, dedifferentiation and growth, altogether resulting in artery maturation. By genetic deletion of a PHD2 allele, monocytes are pre-adapted to an ischemic situation and, as a consequence, monocyte-derived macrophages will be more prone toward an arteriogenic phenotype (and will, e.g., already express SDF-1 and PDGFB). At 72 hours after ligation, this process is initiated in WT mice as indicated by an increase of the mean αSMA⁺ collateral vessel area and infiltration of the adductor with TEMs, but it is not functionally complete since bismuth-perfused collateral area and density were still comparable to the baseline level. Further investigations will be needed to understand if this model plays a role during arteriogenesis in development and if this Tie2-expressing population is similar to the TEM population found in cancer.

Although different proarteriogenic molecules such as MMP2 are up-regulated in PHD2^{+/-} macrophages, SDF 1 and PDGFB were expressed more abundantly. Both cytokines are potent chemoattractants for SMCs and/or SMC progenitors. SDF1 more specifically plays a key role in recruiting, retaining and positioning CXCR4⁺ cells. This might be the case for SMCs and SMC progenitors, both positive for the SDF 1 receptor CXCR4, which can find their way toward collaterals by following a gradient of SDF1 released by pericollateral Tie2-expressing myeloid cells. PDGFB sustains recruitment and proliferation of SMCs and SMC progenitors at the site of expression. In our experiments, only the combined activation of SDF1 and PDGFB achieves a complete formation of collateral branches, suggesting that in SMCs, these two pathways can converge to, at least in part, overlapping downstream effectors.

TEMs are a subpopulation of alternatively activated (M2) macrophages. We show here that the macrophage skewing in PHD2^{+/-} mice is driven by NF-κB activation. The NF-κB family consists of five members: NF-κB1 (p105/p50), NF-κB2 (p100/p52), RelA (p65), RelB, and c-Rel, which may form different homo- and heterodimers associated with differential regulation of target genes. Gene targeting of p50 NF-κB freezes the macrophages in an M1 (proinflammatory) phenotype (Porta et al., Proc. Natl. Acad. Sci. U.S.A. 106:14978-83, 2009). Thus, p50 NF-κB orchestrates the up-regulation of M2-type genes and inhibits the expression of M1-type genes. Here, we found that PHD2 likely breaks this transcriptional cascade; PHD2 down-modulation consistently represses several M1-type cytokines, such as IL12, IL6, IL1β, CXCL10, and up-regulates a specific set of M2-type genes, including Tie2, PDGFB and SDF1.

Finally, our findings have important medical implications. Previous studies have shown that unspecific inhibitors of prolyl hydroxylases (particularly unspecific inhibitors of PHD2) or silencing of PHDs promotes angiogenesis and may thus be beneficial against ischemia (Loinard et al., Circulation 120:50-9, 2009; Milkiewicz et al., J. Physiol. 560:21-6 (2004); Nangaku et al., Arterioscler. Thromb. Vasc. Biol. 27:2548-54 (2007); Huang et al., Circulation 118:S226-33 (2008)). However, this approach can have some limitations. First, angiogenesis is a late response; therefore, organ function might be compromised until new vessel formation is complete. In contrast, arteriogenesis takes place on preexisting vascular shunts and this process is actually the first to be triggered in case of ischemia (Schaper, 2009). Second, the generation of PHD2-specific inhibiting drugs will be challenging due to the high homology of the catalytic pocket of the three PHD family members (PHD1, PHD2 and PHD3). Third, PHD2 can control signaling pathways independently from its enzymatic activity, as is the case for NF-κB regulation; this makes pharmacological inhibitors inefficient. Overall, a cell-based therapy with PHD2 hypomorphic macrophages or Tie2-expressing macrophages might promote collateral vascularization in patients at risk of ischemic damage, i.e., diabetic or hypercholesterolemic patients (Sacco, 1995); similar results may be obtained by the combined administration of SDF1 and PDGFB.

TABLE 5
LIST OF PRIMERS USED FOR QRT-PCR.

| GENE | PROBE | FORWARD | REVERSE |
|---|---|---|---|
| Nos2 | ACT-ATA-ACT-CCA- | AGC-CCG-GGA-CTT- | TGA-AGC-CGC-TGC- |
| | TCA-AAA-GGA-GTG- | CAT-CAA-TC | TCA-TGA-G |
| | GCT-CCC-AG | (SEQ ID NO:4) | (SEQ ID NO:5) |
| | (SEQ ID NO:3) | | |
| Nos3 | ACT-ATA-ACT-CCA- | AGC-CCG-GGA- | TGA-AGC-CGC- |
| | TCA-AAAGGA-GTG- | CTTCAT-CAA-TC | TGCTCA-TGA-G |
| | GCT-CCC-AG | (SEQ ID NO:4) | (SEQ ID NO:5) |
| | (SEQ ID NO:3) | | |
| Icam1 | CCT-CAT-GCA-AGG- | GTC-CGT-GCA-GGT- | CTT-TCA-GCC-ACT- |
| | AGG-ACC-TCA-GCC- | GAA-CTG-TTC | GAG-TCT-CCA-A |
| | T | (SEQ ID NO:7) | (SEQ ID NO:8) |
| | (SEQ ID NO:6) | | |
| Pdgfb | CCC-ATC-TTC-AAG- | CGG-TCC-AGG-TGA- | CGT-CTT-GGC-TCG- |
| | AAG-GCC-ACA-GTG- | GAA-AGA-TTG | CTG-CTC |
| | ACC-T | (SEQ ID NO:10) | (SEQ ID NO:11) |
| | (SEQ ID NO:9) | | |
| Egln1/Phd2 | ACG-AAA-GCC-ATG- | GCT-GGG-CAA-CTA- | CAT-AGC-CTG-TTC- |
| | GTT-GCT-TGT-TAC- | CAG-GAT-AAA-C | GTT-GCC-T |
| | CA | (SEQ ID NO:13) | (SEQ ID NO:14) |
| | (SEQ ID NO:12) | | |
| SFLT1 | TTT-GCC-GCA-GTG- | GAA-GAC-ATC- | TTG-GAG-ATC- |
| | CTCACC-TT-AAC-G | CTTCGG-AAG-CAC- | CGAGAG-AAA-ATG- |
| | (SEQ ID NO:15) | GAA | G |
| | | (SEQ ID NO:16) | (SEQ ID NO17) |
| Cxcl12/Sdf1 | CGG-TAA-ACC-AGT- | CCG-CGC-TT- | GCG-ATG-TGG-CTC- |
| | CAG-CCT-GAG-CTA- | GCAT-CAG-T | TCGAAG-A |
| | CCG | (SEQ ID NO:19) | (SEQ ID NO:20) |
| | (SEQ ID NO:18) | | |
| Tgfβ | TGC-TAA-AGA-GGT- | GAG-CCC-GAA-GCG- | GG-TTG-TTG-CGG- |
| | CAG-CCG-CGT-GCT | GAC-TAC-T | TCC-AC |
| | (SEQ ID NO:21) | (SEQ ID NO:22) | (SEQ ID NO:23) |

For the following genes with sequence ID (enclosed between brackets), commercially available primers were ordered from Applied Biosystems (world wide web at products.appliedbiosystems.com): Ang1 (Mm00456498_m1), Ang2 (Mm00545822_m1), Arg1 (Mm00475991_m1), Calponin-1 (Mm00487032_m1), Ccl17 (Mm00516136_m1), Ccl22 (Mm00436439_m1), Cox2 (Mm00478374_m1), Cxcl1 (Mm00433859_m1), Cxcl10 (Mm99999072_m1), Cxcl2 (Mm00436450_m1), Cxcr4 (Mm01292123_m1), Fizz (Mm00445109_m1), Hgf (Mm01135185_m1, Ifnβ (Mm00439552_s1), IL12α (Mm00434169_m1), IL1β (Mm01336189_m1l), IL6 (Mm01210733_m1), Mcp1 (Mm00441242_m1), Mmp2 (Mm00439506_m1), Mmp9 (Mm00442991_m1), Nrp1 (Mm01253210_m1), NmMHC (Mm00805131_m1), Egln2/Phd1 (Mm00519067_m1), Egln3/PHD3 (Mm00472200_m1), Plgf (Mm00435613_m1), Rantes (Mm01302428_m1), SM22α (Mm00441660_m1), Smoothelin (Mm00449973_m1), Tie2 (Mm00443243_m1), Tnfα (Mm00443258_m1), Vegfα (Mm00437304_m1), Ym1 (Mm00657889_mH), αSMA (Mm01546133_m1), Jagged-1 (MM00496902_m1), VEGFR-2 (MM01222419_m1), Cdh5 (MM00486938_M1).

1. A pharmaceutical composition comprising SDF1 and PDGFB.

2. The pharmaceutical composition of claim 1, further comprising:
an isolated myeloid cell population having increased levels of arteriogenic gene expression as compared to a control myeloid cell population, wherein at least said arteriogenic comprise Tie2, SDF1 and PDGFB.

3. The pharmaceutical composition of claim 2, wherein at least one other arteriogenic gene is expressed in increased amounts, said at least one other arteriogenic gene selected from the group consisting of HGF, TGFb, CXCR4, neuropilin-1, CCR2, Arg 1, FIZZ and MMP2.

4. The pharmaceutical composition of claim 2, wherein the increased arteriogenic gene expression in the myeloid cell population is due to inhibition of PHD2.

5. The pharmaceutical composition of claim 4, wherein the inhibition of PHD2 is by haplodeficiency or acute deletion of PHD2.

6. The pharmaceutical composition of claim 2, together with a bone marrow sample.

7. The pharmaceutical composition of claim 2, wherein the myeloid cell population consists essentially of monocytes.

8. The pharmaceutical composition of claim 2, wherein the myeloid cell population consists essentially of macrophages.

9. (canceled)

10. A method of treating ischemia in a subject, the method comprising:
utilizing the pharmaceutical composition of claim 1 to treat the ischemia in the subject.

11. The method according to claim 10, wherein the ischemia is selected from the group consisting of limb ischemia, muscle ischemia, cardiac ischemia, cerebral ischemia, ischemia in reperfusion injury, liver ischemia, and renal ischemia.

12. (canceled)

13. A method of treating ischemia in a subject, the method comprising:
administering to the subject the pharmaceutical composition of claim 1 to treat the ischemia in the subject.

14. The method of claim 13, wherein administration to the subject is by infusion of monocytes and/or macrophages.

15. The method of claim 13, wherein administration to the subject is by bone marrow transplantation.

16. (canceled)

17. A method of monitoring progression of ischemia in a subject, the method comprising:
taking a sample from the subject;
determining the presence and/or levels of SDF1 and PDGFB in the subject by analyzing the sample; and/or
determining the presence and/or levels of myeloid cells with increased expression of at least Tie2, SDF1 and PDGFB arteriogenic genes in the subject by analyzing the sample, said increased expression as compared to a control myeloid cell population,
wherein increased levels of SDF1 and PDGFB and/or increased levels of myeloid cells with increased expression of SDF 1 and PDGFB correlate with a decrease in ischemia in the subject,
so as to monitor progression of ischemia in the subject and to alter therapy of the subject in view thereof.

18. The pharmaceutical composition of claim 4, wherein increased arteriogenic gene expression in the myeloid cell population is due to partial inhibition of PHD2.

19. The pharmaceutical composition of claim 18, wherein inhibition of PHD2 is by haplodeficiency or acute deletion of PHD2.

20. method of reducing ischemia in a subject, the method comprising:
administering the pharmaceutical composition of claim 1 to the subject, so as to reduce ischemia in the subject.


SEQUENCE LISTING (23 sequences)

SEQ ID NO: 1 - RNA, 24 nt, Artificial Sequence
  siRNA
gccggcugggcaaauacuauguca

SEQ ID NO: 2 - DNA, 24 bp, Artificial Sequence
  siRNA
caccgcttaacccgtattgcctat

SEQ ID NO: 3 - DNA, 32 bp, artificial sequence
  Primer
actataactccatcaaaaggagtggctcccag

SEQ ID NO: 4 - DNA, 20 bp, artificial sequence
  Primer
agcccgggacttcatcaatc

SEQ ID NO: 5 - DNA, 19 bp, artificial sequence
  Primer
tgaagccgctgctcatgag

SEQ ID NO: 6 - DNA, 25 bp, artificial sequence
  Primer
cctcatgcaaggaggacctcagcct

SEQ ID NO: 7 - DNA, 21 bp, artificial sequence
  Primer
gtccgtgcaggtgaactgttc

SEQ ID NO: 8 - DNA, 22 bp, artificial sequence
  Primer
ctttcagccactgagtctccaa

SEQ ID NO: 9 - DNA, 28 bp, artificial sequence
  Primer
cccatcttcaagaaggccacagtgacct

SEQ ID NO: 10 - DNA, 21 bp, artificial sequence
  Primer
cggtccaggtgagaaagattg

SEQ ID NO: 11 - DNA, 18 bp, artificial sequence
  Primer
cgtcttggctcgctgctc

SEQ ID NO: 12 - DNA, 27 bp, artificial sequence
  Primer
acgaaagccatggttgcttgttaccca

SEQ ID NO: 13 - DNA, 22 bp, artificial sequence
  Primer
gctgggcaactacaggataaac

SEQ ID NO: 14 - DNA, 19 bp, artificial sequence
  Primer
catagcctgttcgttgcct

SEQ ID NO: 15 - DNA, 25 bp, artificial sequence
  Primer
tttgccgcagtgctcacctctaacg

SEQ ID NO: 16 - DNA, 24 bp, artificial sequence
  Primer
gaagacatccttcggaagcacgaa

SEQ ID NO: 17 - DNA, 22 bp, artificial sequence
  Primer
ttggagatccgagagaaaatgg

SEQ ID NO: 18 - DNA, 27 bp, artificial sequence
  Primer
cggtaaaccagtcagcctgagctaccg

SEQ ID NO: 19 - DNA, 17 bp, artificial sequence
  Primer
ccgcgctctgcatcagt

SEQ ID NO: 20 - DNA, 19 bp, artificial sequence
  Primer
gcgatgtggctctcgaaga

SEQ ID NO: 21 - DNA, 24 bp, artificial sequence
  Primer
tgctaaagaggtcacccgcgtgct

SEQ ID NO: 22 - DNA, 19 bp, artificial sequence
  Primer
gagcccgaagcggactact

SEQ ID NO: 23 - DNA, 17 bp, artificial sequence
  Primer
gcgttgttgcggtccac